(19) United States
(12) Patent Application Publication
(10) Pub. No.: US 2026/0115676 A1
(43) Pub. Date: Apr. 30, 2026

(54) SMART COMPOUND PREPARATION SYSTEM WITH POTENCY ESTIMATION, MODULAR COMPONENTS, AND GUIDED WORKFLOW INTEGRATION

(71) Applicant: Dennis Wysocki, Middletown, DE (US)

(72) Inventor: Dennis Wysocki, Middletown, DE (US)

(21) Appl. No.: 19/425,642

(22) Filed: Dec. 18, 2025

Related U.S. Application Data
(63) Continuation-in-part of application No. 17/196,500, filed on Mar. 9, 2021, now abandoned.

(51) Int. Cl. B01F 35/22; B01F 23/41; B01F 27/112; B01F 35/00; B01F 35/90; B01F 101/06; B01F 101/21

(52) U.S. Cl. CPC B01F 35/2206; B01F 23/41; B01F 27/112; B01F 35/561; B01F 35/90; B01F 2035/99; B01F 2101/06; B01F 2101/21

(57) ABSTRACT

A compound preparation system for infusing, melting, activating, or blending botanical, fungal, or carrier formulations using indirect heat, programmable mixing, and compound-specific logic. The system integrates a removable canister, sensor-driven thermal control, modular agitation, and a processor executing workflows based on the ELOH (Empirical Logic Organizing Heuristics) database. The system enables users to optimize compound yield, flavor, scent, or potency through guided prompts, real-time estimation, and synergy-aware enhancements.

### CROSS-REFERENCE TO RELATED APPLICATIONS

The present application is a Continuation-In-Part of and claims priority to U.S. Non-Provisional patent application Ser. No. 17/196,500 filed on Mar. 9, 2021, entitled “HERBAL DECARBOXYLATION AND INFUSION SYSTEM” the entire disclosure of which is incorporated by reference.

### TECHNICAL FIELD

The present invention relates to compound preparation systems for producing infused, activated, or blended formulations. More particularly, it pertains to smart systems for preparing nutrient-rich components, such as botanical extracts, functional infusions, and therapeutic blends, for integration into culinary, wellness, or topical workflows. The invention is especially suited for enhancing bioavailability and preserving the stability of heat-sensitive compounds through precise thermal control, guided workflows, and modular processing.

### BACKGROUND

In various industries ranging from culinary arts to personal care and wellness, there exists a growing demand for high-quality infused or blended products incorporating functional ingredients into liquid or semi-solid carriers. These products often require precise control over temperature, mixing, and timing to ensure that active compounds are extracted, dissolved, or dispersed effectively without compromising their stability or efficacy. However, conventional preparation methods, including stovetop infusion, double boilers, or manual blending, typically lack the precision and repeatability necessary to achieve consistent results.

Many existing appliances rely on direct-contact heating elements with minimal or no feedback control, leading to common issues such as localized overheating, uneven compound distribution, and degradation of heat-sensitive ingredients. Additionally, such methods are highly dependent on user intervention, experience, and guesswork. This not only increases the likelihood of user error but also results in inconsistencies between batches, particularly when dealing with volatile, aromatic, or biologically active compounds.

Furthermore, traditional infusion and mixing devices are typically limited in scope and functionality. For instance, a slow cooker may provide basic heating, but it cannot control agitation, measure compound potency, or guide the user through complex, multi-step preparation sequences. Similarly, countertop blenders and mixers may offer mechanical agitation but are not equipped to handle controlled heating, staged cooling, or ingredient-specific processing profiles. These limitations make such devices ill-suited for the precise demands of modern culinary and formulation science.

Formulating infused oils, syrups, balms, creams, or emulsions often involves managing multiple thermally sensitive stages, such as melting solid carriers, dissolving powdered actives, integrating liquids of differing viscosities, and homogenizing ingredients into a uniform end product. For example, preparing a topical balm may require melting a blend of butters and waxes, introducing botanical infusions at lower temperatures, and then blending the mixture before it cools. General-purpose tools cannot manage these transitions in a coordinated or intelligent manner, leading to poor texture, separation, or reduced compound integrity.

Another significant shortcoming of existing systems is the lack of compound-aware intelligence. Most appliances do not account for the solubility, degradation thresholds, or volatility of specific ingredients, nor do they adapt their behavior based on carrier selection. As a result, users must rely on external references or trial-and-error to determine infusion times, temperatures, and mixing strategies. This not only reduces efficiency but also increases material waste and reduces the therapeutic or nutritional value of the final product.

Moreover, very few systems provide real-time feedback or dynamic control based on live sensor data. Without integrated temperature or mixing sensors, appliances cannot self-correct or compensate for external variables such as ambient temperature, ingredient volume, or carrier density. Consequently, even when the same input materials are used, outcomes can vary significantly from batch to batch. Inconsistent preparation undermines product quality, consumer trust, and, in regulated environments, compliance with labeling or dosage requirements.

The growing popularity of functional formulations, including infused cooking oils, therapeutic balms, nutritional syrups, and scent-forward products like candles and aromatherapeutic oils, has highlighted the need for more advanced preparation platforms. Consumers and professionals alike seek greater control, safety, and automation in preparing such products. They require systems that not only execute precise thermal and mechanical tasks but also guide the user through complex workflows with compound-specific intelligence.

While some multi-function kitchen appliances or cosmetic mixers attempt to fill this gap, they typically offer only limited preset routines, lack remote or mobile integration, and are not adaptable to a wide range of ingredient-carrier combinations. These systems generally do not support staged, multi-phase processes, nor do they provide integrated guidance or potency estimation tools. As such, they are insufficient for users who demand repeatability, flexibility, and the ability to prepare a variety of custom or small-batch infused formulations.

There is therefore a need for a modular, intelligent, and compound-aware system that can execute programmable workflows for a wide range of infusion, melting, mixing, and activation tasks. Such a system should provide indirect, sensor-controlled heating to protect sensitive ingredients, support active mixing strategies tailored to viscosity and formulation goals, and offer real-time user guidance through a local or mobile interface. It should also incorporate logic-driven workflow management based on ingredient properties, desired outcomes, and process history.

The present invention addresses these needs by introducing a smart compound preparation system capable of managing thermally and mechanically sensitive workflows with minimal user intervention. Through integrated heating, mixing, sensing, and logic control, the system enables users to prepare high-quality formulations with consistency and safety. Whether used for culinary infusions, wellness supplements, cosmetic balms, aromatherapeutic products, or laboratory-scale formulations, the system delivers process precision, batch traceability, and adaptive user interaction previously unavailable in consumer-grade or small-batch preparation devices.

### SUMMARY OF THE INVENTION

The invention is a compound preparation system configured to execute modular, programmable workflows involving organic materials and carrier substances. It enables users to perform infusion, activation (e.g., decarboxylation), melting, and mixing through a unified platform that combines indirect heat, real-time sensor feedback, and staged process control. A removable, thermally isolated canister supports both standalone and sequenced operations, facilitating preparation of infused oils, balms, waxes, syrups, and other functional blends with minimal intervention. By automating time-and temperature-sensitive tasks, the system helps preserve compound potency, improve bioavailability, and deliver consistent formulation outcomes across culinary, therapeutic, and cosmetic applications.

The present invention provides a universal compound preparation platform designed for the controlled infusion, melting, activation, and blending of a wide variety of ingredients into diverse solvent or carrier mediums. The system supports virtually any ingredient type, whether botanical, synthetic, aromatic, nutritional, or functional, and accommodates a range of carriers including oils, waxes, glycerin, water, syrups, alcohols, emulsions, and other solvents. Through programmable heating, mixing, and process sequencing, the apparatus enables users to create customized formulations for culinary, cosmetic, wellness, industrial, or laboratory applications with minimal manual oversight. The system's closed-loop temperature regulation, modular mixing mechanisms, and database-driven logic ensure that ingredient integrity, solubility, and volatility are maintained throughout each preparation cycle. Whether producing flavor-infused oils, therapeutic creams, scented waxes, functional beverages, or encapsulated supplements, the system offers a highly adaptable solution for consistent, scalable, and safe ingredient-solvent integration.

The system is controlled through a touchscreen interface embedded in the housing, providing access to presets, user profiles, live metrics, and customizable workflows. A companion mobile application, connected via Wi-Fi, mirrors this interface and enables remote interaction. Both are governed by the ELOH (Empirical Logic Organizing Heuristics) control framework, which manages temperature, timing, agitation, potency estimation, and adaptive user guidance. This guidance delivers real-time, context-aware prompts, such as alerts when it is safe to pour, cooling recommendations, or handling warnings, to preserve compound integrity and guide safe user handling. ELOH also supports firmware updates, remote recipe uploads, and ongoing system optimization.

The system executes compound preparation through coordinated stages, which may include: melting of solid or semi-solid materials, thermal activation of precursors, infusion of bioactive compounds into a carrier, and active mixing for dispersion or emulsification. These stages can be performed independently or as part of user-defined sequences, with thermal input regulated by closed-loop logic based on external sensor data. Process profiles may be tailored to material properties, compound sensitivity, or formulation goals, ensuring consistent formulation even for heat-sensitive or volatile ingredients.

The removable canister, constructed from food-safe, heat-resistant materials, such as stainless steel, borosilicate glass, or other suitable materials, functions as a modular processing chamber with interchangeable blade or blade-free configurations for mixing, emulsifying, or homogenizing botanical, fungal, or carrier materials. Coupled with an indirect heating system (radiant or conductive, at a maximum temperature suitable for compound integrity, e.g., 300° F./149° C.), it enables preparation tasks akin to a blender in a controlled oven. Sensor-regulated feedback supports accurate control, preserving compounds like essential oils or polysaccharides. A sealed lid with a silicone plug facilitates low-odor processing.

Sensor-regulated thermal feedback maintains uniform heat distribution and prevents localized overheating, preserving thermally sensitive constituents such as essential oils, cannabinoids, and mushroom polysaccharides. Integrated cooling fans expedite cooldown between operations, reducing cycle time and mitigating compound degradation. A sealed lid with a silicone plug reduces vapor escape and supports passive odor control, enabling low-odor processing of volatile or aromatic inputs for home or small-batch use.

The system supports various batch sizes, including small to medium batches, depending on canister design, suitable for home and small-scale commercial applications. Interchangeable canisters, firmware updates, and accessory integration enable adaptation across culinary, functional, and aromatic workflows, preserving compound integrity. By coordinating thermal and mechanical operations with external task guidance, the system facilitates repeatable outcomes.

In some embodiments, the system is configured to support infusion routines beyond cannabis decarboxylation, including the incorporation of botanical, fungal, or nutritional compounds into various carriers such as oils, glycerin, syrups, honeys, and alcohols. These workflows are guided by stored thermal and mechanical profiles tailored to each carrier's viscosity, compound solubility, and volatility. By combining indirect heating and programmable agitation, the system allows for the controlled infusion of both hydrophilic and lipophilic compounds, optimizing yield and consistency across applications.

The system also supports programmable mixing operations for emulsification, homogenization, or maceration workflows. This enables users to blend immiscible components such as oil and water or integrate heat-sensitive actives into cooled carriers. Through pulse or continuous agitation modes, selected via touchscreen or mobile app, the system promotes even distribution of actives within high-viscosity or phase-sensitive media. Applications may include infused balms, whipped butters, culinary emulsions, and topical creams.

In further embodiments, the apparatus enables the use of encapsulating agents such as lecithin or agar to form compound-containing vesicles or micelles for enhanced delivery or shelf stability. These encapsulation workflows may involve controlled temperature ramps and mixing cycles, enabling formation of microstructures without compound degradation. The processor references stored parameters to ensure thermal compatibility between carrier, encapsulant, and active material, enhancing product integrity.

The system may also execute sequential or multi-phase workflows wherein different compounds are infused at staggered intervals. This functionality is particularly valuable for layered formulations, such as combining relaxing and energizing botanicals in distinct phases, or integrating scent and therapeutic actives into dual-use products. Intermediate cooling, filtration, or mixing steps may be guided through the interface, with adaptive timing and alerts to ensure process fidelity.

In some applications, the system enables controlled scent infusion into wax or oil carriers for aromatherapeutic candles, massage oils, or fragrance preparations. The processor references compound-specific temperature limits for volatile scent compounds and supports addition of stabilizers or fixatives to enhance fragrance retention. Modular canisters with sealed lids and vapor control options enable low-odor operation and directed scent retention.

These expanded capabilities demonstrate the system's suitability for diverse workflows beyond decarboxylation. By combining sensor-regulated thermal logic, modular mixing hardware, and compound-aware guidance, the system supports precision preparation of culinary, wellness, topical, and aromatic formulations. Its programmable flexibility allows advanced users to customize recipes while providing novices with guided presets for safe and repeatable results.

### BRIEF DESCRIPTION OF THE DRAWINGS

A complete understanding of the embodiments and the associated advantages and features will be more readily understood by reference to the following detailed description when considered in conjunction with the accompanying drawings, wherein:

FIG. 1 illustrates a block diagram of the system, according to some embodiments;

FIG. 2 illustrates a flowchart of a method of use of the apparatus, according to some embodiments;

FIG. 3 illustrates a perspective view of the decarboxylation and infusion apparatus, according to some embodiments;

FIG. 4 illustrates a perspective view of the decarboxylation and infusion apparatus, according to some embodiments;

FIG. 5 is a cross-sectional view of the decarboxylation and infusion apparatus, illustrating internal components including the mixing element, heating reservoir, ventilation system, and insulation layer, according to some embodiments;

FIG. 6 is a block diagram of the network infrastructure of the infusion system, illustrating connections between the apparatus, mobile application, cloud database, and user computing devices, according to some embodiments;

FIG. 7 illustrates a perspective view of the decarboxylation and infusion apparatus, including the removable processing chamber, user interface, and integrated lid mechanism;

FIG. 8 illustrates a perspective view of the decarboxylation and infusion apparatus, showing the handle, spout alignment mechanism, and removable canister in a disengaged or lifted state, according to some embodiments;

FIG. 9 illustrates a perspective view of the decarboxylation and infusion apparatus, highlighting the sealed lid configuration, top-mounted handle, and locking mechanism, according to some embodiments;

FIG. 10 illustrates a flowchart of a method for initiating an infusion process using the smart infusion system. The flow includes user selection of a compound type and intended use (e.g., culinary, topical), followed by touchscreen-guided selection of a carrier medium (e.g., oil, honey), activation mode, and process intensity. Each step includes real-time feedback and confirmation prompts to reduce user error and reinforce safe handling during heating and mixing;

FIG. 11 illustrates a flowchart of a method for estimating potency based on input variables such as material type, weight, carrier volume, carrier absorption, and serving size, including correction factors such as decarboxylation efficiency and carrier absorption,

FIG. 12 illustrates a block diagram of a computing system configured to execute the application program associated with the smart infusion system;

FIG. 13 illustrates a screenshot of the user interface showing selectable modes such as “Infuse,” “Melt,” “Mix,” “Activate,” “Calc,” and “Settings,” each linked to process parameters;

FIG. 14 illustrates a schematic view of the application's multi-screen navigation interface, showing various infusion targets and associated settings;

FIG. 15 illustrates a block diagram of the application architecture, including modules for communication, database, display, user interaction, potency estimation, and alerts;

FIG. 16 illustrates a flowchart of a guided compound preparation method with internal and external coordination;

FIG. 17 illustrates a database schema for the ELOH system, showing the interrelation of compound, carrier, function, user input, and guidance tables used to support the system's operational logic, according to some embodiments;

FIG. 18 illustrates an exemplary coordination interface between a mobile device and the ELOH system, guiding the user through a multi-step infusion process that may involve external appliances, according to some embodiments; and

FIG. 19 illustrates a feedback loop architecture in which the ELOH system collects post-process user ratings and session data to inform future presets, firmware behavior, and recipe optimizations, according to some embodiments.

### DETAILED DESCRIPTION

The system integrates modular hardware (removable canisters, interchangeable mixing assemblies) with real-time control logic and a touchscreen interface to execute low-temperature workflows for botanical, fungal, or carrier formulations. It automates infusion, activation, melting, and homogenization, guided by sensor feedback and the ELOH framework, supporting repeatable results. In some embodiments, it coordinates multi-step workflows with external appliances, using user feedback to refine settings.

The present embodiments are based in part upon extraction procedures and delivery approaches that allow selective utilization of various cannabinoid molecules and terpenes from the cannabis plant. These various cannabinoid compounds are designed to selectively affect various cannabinoid receptors in the nervous system, immune system, and other tissues. The extract is an oil-based or solvent-based plant product that contains inactive and active compounds contained in the cannabis plant such as cannabinoids, terpenes, and/or flavonoids. Compositions of the invention and methods of extraction disclosed herein provide an extract with specific physiological properties that are mediated through separate pathways and receptors, which provide numerous benefits and advantages.

The system described herein is broadly applicable to the preparation of infused, blended, melted, or activated formulations involving any ingredient and solvent combination where thermal control, compound integrity, and process repeatability are desired. The apparatus supports workflows for culinary, cosmetic, wellness, industrial, or laboratory applications by enabling precise heating, agitation, and timing parameters to be applied to virtually any material, solid, semi-solid, or liquid, and any solvent or carrier, including oils, waxes, glycerin, water, alcohols, syrups, or emulsions. Whether the goal is to dissolve a powdered active, infuse a flavor compound, emulsify immiscible liquids, melt and pour a wax base, or homogenize a formulation, the system delivers repeatable outcomes through sensor-driven feedback and programmable control logic. Ingredients may include natural extracts, synthetic compounds, essential oils, flavoring agents, surfactants, gelling agents, fragrances, or colorants. Carriers may be selected based on solubility, thermal compatibility, or end-use application, with the system guiding parameter selection to avoid overheating, under-infusion, or compound degradation. These capabilities allow the apparatus to serve as a universal platform for controlled ingredient-solvent processing across a wide spectrum of industries and formulation goals.

The embodiments described herein relate not only to decarboxylation but to a broader category of compound preparation processes involving the infusion, blending, activation, and dispersion of botanical, fungal, and functional compounds into a variety of carrier mediums. The system is designed to serve as a flexible platform for preparing compound-rich formulations that may be culinary, therapeutic, cosmetic, or aromatic in nature. This includes the creation of infused oils, balms, emulsions, syrups, waxes, and encapsulated formulations using programmable heating and mixing routines governed by compound-specific logic.

Unlike conventional single-function appliances, the disclosed system combines indirect heat regulation, modular hardware, and software-driven process coordination to execute complex workflows with minimal user intervention. These workflows may include emulsification of oil and water phases, stabilization of scent or flavor compounds, infusion into viscous carriers like honey or glycerin, or precision mixing of temperature-sensitive bioactives. Through sensor-driven control and guided user prompts, the system ensures uniformity, potency, and repeatability across diverse ingredient and carrier combinations.

In addition to infusing compounds into liquid or semi-solid carriers, the system is also capable of supporting encapsulation workflows, wherein actives are combined with structural or delivery agents like lecithin, agar, or wax to form micelles, beads, or protective coatings. These processes often require exact thermal staging and dynamic mixing profiles to avoid degradation while achieving encapsulation fidelity. The apparatus enables such sequences through modular programming, adjustable agitation profiles, and real-time thermal feedback.

The detailed description that follows provides an overview of the system's components, operational logic, and interface structure, with particular attention to the methods and mechanisms by which the apparatus performs these expanded workflows. Though some examples reference cannabinoid activation, the described functionality is broadly applicable to a range of botanical and functional materials where compound preservation, accurate dosing, and workflow coordination are required.

In one embodiment, the system is configured to infuse turmeric root into a lipid-based carrier such as coconut oil or ghee. The user selects a preset infusion profile associated with curcumin, the active compound in turmeric, and the system regulates a low and sustained temperature (e.g., 140° F. for 2 hours) to avoid oxidative degradation. The user may optionally introduce piperine, a known bioavailability enhancer derived from black pepper, at a specified time point guided by system prompts. The mixing element performs intermittent agitation throughout the cycle to ensure even curcumin distribution and prevent sedimentation. The final product is a nutraceutical-grade infused oil suitable for ingestion, topical application, or encapsulation.

In another embodiment, the system facilitates the preparation of an arnica-infused balm for topical use. The user loads dried arnica flowers and a beeswax-coconut oil mixture into the removable canister, selecting the “Topical Balm” workflow from the touchscreen interface. The processor initiates a two-phase heating sequence, first melting the carrier blend at approximately 150° F., followed by a lower-temperature infusion phase at 130° F. with timed mixing intervals. Once complete, the user is prompted to pour the formulation while still molten, allowing it to cool and solidify into a balm. This sequence preserves the therapeutic integrity of arnica's sesquiterpene lactones while achieving uniform dispersion in the semi-solid base.

In some embodiments, the system may be used to produce culinary infusions such as garlic- or rosemary-infused olive oil. The user selects a “Culinary Infusion” mode and is prompted to input the botanical material type and desired flavor strength (e.g., mild, balanced, bold). The system references stored aromatic volatility thresholds and executes a low-heat infusion cycle, typically between 120° F. and 140° F., to extract flavor compounds without scorching. Mixing cycles are gentle and spaced out to avoid mechanical breakdown of delicate herbs. The result is a flavor-infused oil with enhanced shelf stability and consistent potency across batches.

In a further embodiment, the apparatus is configured to prepare infused honey with ginger and lemon. The user selects a “Functional Syrup” mode and introduces fresh ginger slices and lemon zest into the chamber along with a volume of raw honey. The system initiates a gentle warm-up phase to reduce the viscosity of the honey and facilitate infusion without compromising its enzymatic properties. Agitation is performed using a low-speed mixing profile to avoid incorporating air or causing crystallization. Once complete, the system prompts the user for optional straining and outputs a ready-to-use infused syrup suitable for wellness or culinary purposes.

In cosmetic applications, the system may be employed to produce a shea butter-based body cream infused with calendula and chamomile. A two-stage process is used: first, the carrier blend (shea butter and jojoba oil) is melted and homogenized at approximately 160° F. Second, dried flower material is introduced, and the temperature is reduced to 130° F. to preserve active flavonoids. Intermittent mixing ensures even compound distribution without excessive shear. Upon completion, the system activates its cooling cycle to bring the formulation to a pourable temperature for mold-filling or jar packaging.

In yet another embodiment, the system supports preparation of scented soy wax candles. The user selects a “Candle” preset and loads soy wax pellets into the canister, which are melted at a controlled 130-140° F. range depending on wax type. Once fully liquefied, the system prompts the user to add fragrance oils or essential oils such as lavender, eucalyptus, or sandalwood. The processor references the compound's scent-retention temperature threshold and adjusts mixing and heat exposure to maximize aroma preservation. The user is then guided through pouring into molds or containers, with real-time alerts on optimal pouring temperature to avoid frosting or sinkholes in the final product.

In one embodiment related to functional beverages, the system facilitates infusion of powdered adaptogens such as ashwagandha or maca root into a dairy or plant-based milk. The system heats the base liquid to a user-defined target (e.g., 120° F.) and slowly integrates the powdered ingredient using programmed agitation to prevent clumping or sedimentation. A shear-emulsification mode may be employed depending on the ingredient's solubility characteristics. This results in a shelf-stable, uniformly dispersed beverage concentrate with enhanced mouthfeel and absorption. The infusion parameters can be stored for repeat batches or scaled to larger volumes using interchangeable canisters.

In another non-culinary embodiment, the system is used to prepare a mushroom-based wellness extract using dried reishi and chaga mushrooms. The system initiates an aqueous infusion cycle with a temperature of approximately 180° F., sustained over multiple hours to extract polysaccharides and beta-glucans. Optional transformative agents such as citric acid may be added to enhance extraction efficiency. The mixture is then filtered and optionally infused into a lipid carrier such as MCT oil for improved bioavailability. The resulting dual-extracted tincture may be used in dietary supplements, wellness elixirs, or sublingual applications.

In some embodiments, the apparatus may be used to formulate emulsified dressings or culinary sauces. For example, the system may prepare a garlic aioli by blending egg yolk, mustard, and oil at a low speed, then introducing minced garlic and lemon juice at predefined intervals. The system's precise thermal regulation ensures the preparation remains within safe handling temperatures while maintaining a stable emulsion. The final product exhibits consistent texture, flavor, and shelf life, and may be poured directly from the canister into containers or dispensers.

In a final alternative embodiment, the system is configured to perform standalone carrier preparation workflows such as melting cocoa butter, deodorizing oils, or conditioning beeswax prior to active infusion. The user selects a “Melt Only” mode and adjusts temperature and duration manually or via preset. The canister maintains the carrier material in a liquefied state while minimizing exposure to oxygen or unnecessary agitation. These workflows allow users to create neutral bases or custom blends that can later be infused, scented, or colored using additional modules or manual steps.

The extracts and/or delivery methods of the embodiments allow a wide range of prevention, treatment, and management options for patients. In some aspects, the delivery methods of the invention allow for employing micro-dosing with a stacking method of cannabinoid administration week-by-week until a certain saturation point, which is based on the response, weight, and monthly-quarterly test results. One skilled in the arts will readily understand the variety of product configurations and delivery mechanisms that may be produced using the embodiments.

It has been found that the age of the cannabis plant material in addition to the temperature in which it is stored and processed is critical. Importantly, for an extract to produce psychoactive properties or other significant properties found as a result of the consumption of decarboxylated molecules, the cannabis plant material is heated above 160° F.

Further the embodiments provided herein relate to a decarboxylation and infusion apparatus to produce an infused solvent. The apparatus decarboxylates organic material such as cannabis to activate molecules contained in the plant material. In one example, the apparatus is used to decarboxylate Cannabidiolic acid (CBDA) and Tetrahydrocannabinolic acid (THCA) into Cannabidiol (CBD) and Tetrahydrocannabinol (THC). One skilled in the arts will appreciate that various molecules contained in cannabis will readily undergo similar decarboxylation.

To facilitate decarboxylation, the apparatus is comprised of a heating element that can be selectively programmed to heat a reservoir containing the organic material to a specific temperature or temperature range. The heating element is further utilized to facilitate the infusion of the decarboxylated molecules into a solvent. It is known that infusion of molecules, including CDB and/or THC, is accomplished in a solvent such as cooking oils, glycerin, butter, or alcohol.

In some embodiments, the apparatus is in operable communication with a computing device that allows the user to control the function and operational settings of the apparatus during use. A mobile app may be downloaded to the computing device having a processor configured to perform instructions stored in a database. The database can include operational settings such as decarboxylation and infusions times, temperatures, pressures, agitation cycles, and protocols.

In some embodiments, the database may include a list of decarboxylation and infusion protocols for various recipes. Each recipe may be specific to one or more molecules, one or more organic materials, one or more solvents, and combinations thereof. Selecting a recipe may cause the apparatus to autonomously execute the instructions thereof.

The embodiments disclosed herein provide a comprehensive, intelligent, and modular system for performing the controlled infusion of organic materials into various solvent mediums. The system comprises both hardware and software components that collectively perform heating, mixing, user guidance, and data-driven analysis. The apparatus is configured to allow novice and expert users alike to produce consistent infused products with minimal intervention or technical training. The infusion system is housed within a durable enclosure and is operably connected to a user interface, a mobile application, and several integrated modules to control functionality, safety, and data feedback.

In one aspect, the smart infusion system includes a computing device such as a smartphone, tablet, or other remote processing device that runs a dedicated mobile application. The mobile application is configured to serve as an extension of the onboard user interface, allowing the user to control the infusion apparatus wirelessly. The application may be downloadable from a digital distribution platform and, upon installation, establish secure wireless communication with the apparatus using Bluetooth, Wi-Fi, or other similar communication protocols. This connection allows real-time command and feedback between the mobile device and the apparatus throughout the infusion cycle.

The mobile application includes a graphical user interface (GUI) through which a user can input infusion preferences. These preferences may include organic material type, solvent selection, desired infusion potency, decarboxylation temperature, time duration, agitation level, and cooling preferences. The application may offer selectable preset profiles, user-defined profiles, or guided prompts for novice users. The interface may also permit users to store custom profiles, set scheduled start times, and log past infusion sessions for tracking and quality control.

In some embodiments, the mobile application transmits infusion commands and profile parameters to the main controller of the infusion system. The system receives these parameters and automatically configures internal components, including heating elements, sensors, and mixing blades, in accordance with the user's input. The mobile application may additionally provide a progress indicator for each phase of the process, such as pre-heating, decarboxylation, infusion, cooling, and cleaning. Notifications and alerts may be delivered via push messages to the mobile device in real-time.

A key component of the system is the potency estimation module, which calculates the expected concentration of one or more bioactive compounds (e.g., cannabinoids, terpenes, or flavonoids) in the resulting infused solvent. The potency estimation module may reside locally within the apparatus processor or may be cloud-based and accessed via the mobile application. This module uses a variety of inputs including the weight of the organic material, its classification or strain, the selected solvent, the heating duration, the decarboxylation temperature, and the agitation profile.

In some embodiments, the potency estimation module references a database of empirical data or models that correlate infusion parameters with expected compound yields. The module may employ regression models, lookup tables, or machine learning algorithms trained on historical infusion outcomes. The potency estimate may be updated dynamically throughout the infusion process as the system records live temperature data and agitation events. This enables a real-time recalculation of expected potency should any deviations in process conditions occur.

The user may view the estimated potency through either the onboard user interface or the mobile application. The displayed values may include concentration in milligrams per milliliter (mg/mL), percentage by weight, or estimated dosage per unit volume. In some embodiments, the user may be prompted to input additional optional information such as strain age, moisture content, or previous storage conditions to improve the accuracy of the calculation. The system may optionally allow users to export potency data for labeling or recordkeeping purposes.

The system also includes an auto-cleaning module to simplify post-infusion maintenance. The auto-cleaning module is in operable communication with the system processor and includes a cleaning solution reservoir, spray nozzles or rinse ports, and a heating element for sanitation. Following completion of the infusion cycle, the user may select a cleaning mode from the interface, or the system may initiate cleaning automatically based on a predefined protocol.

During cleaning, the heated reservoir or processing chamber may be flushed with water, alcohol-based cleaner, or other cleaning agents. The system agitates the cleaning fluid using the integrated mixing mechanism to ensure contact with all internal surfaces. The liquid is then evacuated via a drain port or collected in a disposable cleaning tray. The system may perform one or more rinse cycles until temperature or turbidity sensors confirm acceptable cleaning levels.

In certain embodiments, the cleaning module also sanitizes external-facing surfaces of the chamber or lid using steam or UV light sources. This additional sanitation stage is particularly useful in applications involving therapeutic-grade or ingestible materials. The cleaning module may be programmed to operate on a fixed schedule, after a set number of cycles, or only when selected by the user. Cleaning status and completion are communicated to the user via the touchscreen or mobile application.

An alert module is provided to ensure safe and informed user interaction with the infusion process. The alert module includes one or more output devices such as speakers, LEDs, vibration motors, or wireless communication components. Alerts may be triggered by key events such as process completion, user input confirmation, system errors, or changes in chamber conditions. In some embodiments, the alert module is also capable of announcing audible instructions or displaying on-screen prompts as part of the guided infusion system.

The alert system may interface with external devices or networks to forward notifications to remote users. For example, alerts may be sent via email, text message, or push notification to the mobile application. The system may allow users to configure the frequency, type, and delivery method of alerts via the settings menu. In safety-critical events such as overheating, open-lid detection, or electrical faults, the alert module may also activate emergency shutdown procedures to prevent damage or injury.

The infusion system utilizes a guided infusion module configured to walk the user through multi-step infusion procedures. These procedures may include decarboxylation, solvent addition, agitation stages, infusion temperature ramps, and cooling or storage prompts. The guided module is operable to restrict advancement through each stage until the previous step is confirmed as complete. The system may utilize lid sensors, weight sensors, and user confirmation buttons to verify step completion.

In one aspect, the guided infusion module uses predictive algorithms to dynamically adjust infusion instructions based on real-time sensor data and user input. For example, if the target potency has not yet been reached by the end of the expected duration, the system may recommend additional time or agitation cycles. Conversely, if the temperature exceeds the optimal threshold early, the module may prompt the user to shorten the process or reduce heating levels. These real-time suggestions help ensure a consistent product even in the presence of minor variations in material or ambient conditions.

In a preferred embodiment, the system incorporates a closed-loop thermal control mechanism in which one or more temperature sensors are in continuous communication with the central processor to prevent the heating element from exceeding a predefined threshold temperature associated with a specific ingredient or compound. The processor references the compound's thermal sensitivity profile, which may be stored in the ELOH database, and establishes an upper temperature limit prior to initiating the infusion or melting cycle. As the canister temperature approaches this limit, the processor dynamically adjusts the power supplied to the heating element, either modulating or pausing heat application entirely to maintain a safe thermal range. This sensor-driven feedback loop operates in real time, sampling temperature data at regular intervals, such as every 500 milliseconds, to ensure compound integrity is preserved. If the temperature rises beyond an acceptable variance, the system may trigger visual or audible alerts, initiate an automatic cool-down cycle, or pause the infusion sequence until safe conditions are restored. This preferred embodiment is especially critical for heat-sensitive compounds such as essential oils, fat-soluble vitamins, and aromatic terpenes, where even brief exposure to excess heat can lead to degradation or loss of efficacy. By coupling real-time sensor monitoring with compound-aware logic, the system ensures safe, repeatable, and high-fidelity preparation across a wide range of formulation types.

The processor unit responsible for controlling the apparatus is configured to execute operational commands and interface with all sensor inputs and module outputs. The processor may be embedded in the main housing or distributed across subcontrollers depending on system architecture. In some embodiments, the processor includes memory for storing operating instructions, infusion data logs, and user preferences. Communication between processor components and the user interface is managed through a software abstraction layer or application programming interface (API).

The apparatus further includes a database that stores a library of infusion profiles and settings. The database may reside in local memory, within the mobile application, or in a cloud-based environment accessible by registered users. Each infusion profile in the database may be tagged with descriptors such as compound type, use-case category, or recommended dosage. Users may retrieve, modify, or upload profiles via the user interface or mobile application. This database system allows for ongoing expansion of available recipes and protocols.

The infusion system may optionally include a learning module configured to track user preferences and refine infusion results over time. The module uses data from completed sessions, such as duration, temperature logs, compound yield estimates, and user feedback, to refine future recommendations. In one embodiment, the learning module generates modified versions of past infusion profiles to enhance performance or accuracy based on historical outcomes. This adaptive process creates a personalized system experience optimized for each user's materials and goals.

The system may also include a remote diagnostics module that allows technicians or developers to access system logs, error reports, and sensor data for troubleshooting and performance enhancement. This module is accessible only with user permission and may be used for warranty service, software updates, or feature testing. Secure authentication protocols are implemented to protect user data and prevent unauthorized access. Remote diagnostics further enhance product reliability and maintainability.

Additional features of the system may include a modular lid with integrated locking mechanisms, safety interlocks for high-temperature conditions, and overload protection for mixing components. The housing of the apparatus may be constructed from heat-resistant polymers, stainless steel, or food-safe materials as required by the intended application. The system may be designed for countertop use, portable deployment, or integration into a larger production line for commercial manufacturing.

The various components described herein may be manufactured, assembled, and calibrated using standard industrial or consumer electronics methods. Electrical connections between elements may include flexible printed circuits, wire harnesses, or contact pads. Plumbing components for cleaning and drainage may include silicone tubing, one-way valves, or peristaltic pumps. The components are arranged to maximize system compactness, ease of maintenance, and mechanical robustness.

The system may also support multiple user accounts or permission tiers to control access to certain functions. For example, a parent or supervisor account may configure and lock advanced settings, while general users may only access profile selection and basic monitoring. In commercial deployments, this allows operators to standardize results while limiting procedural errors. User permissions may be managed through the mobile application or a web-based administrative portal.

In alternative embodiments, the infusion system may support direct integration with smart home systems, laboratory automation platforms, or inventory management databases. For example, the apparatus may sync with a smart speaker for voice control or notify a lab database upon profile completion. The open-ended architecture and wireless compatibility make the system extensible for future integration with external software and services. These features make the system suitable for both standalone and ecosystem deployments.

Taken together, these features provide a novel, user-friendly, and highly customizable infusion platform. The system promotes accuracy, safety, repeatability, and data traceability in the production of infused products. The modular components, smart software, and robust hardware design work in harmony to deliver an unmatched user experience across consumer, clinical, and commercial applications. The components described above may be further understood with reference to the accompanying drawings and the claims that follow.

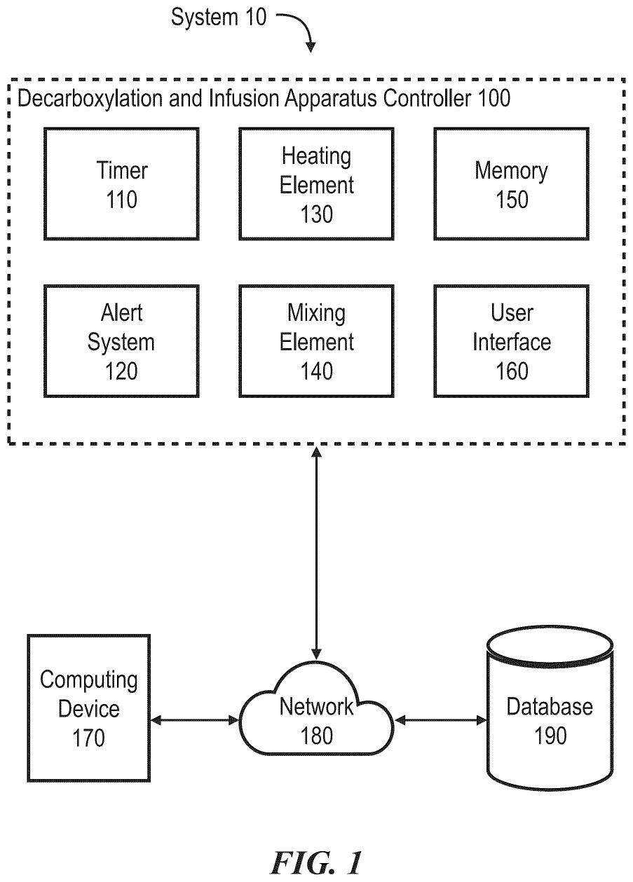

In reference to FIG. 1, the system 10 for decarboxylating and infusing organic materials includes a decarboxylation and infusion apparatus controller 100 configured to produce a usable infused product which may be ingested or otherwise delivered to the user. The apparatus controller 100 may operate without the use of auxiliary tools or appliances. The apparatus controller 100 is in operable communication with a timer 110, alert system 120, heating element 130, and mixing element 140, which are programmable to carry out procedures for decarboxylating and infusing organic materials. The heating element 130 provides heat to a reservoir wherein the organic material is decarboxylated and infused. The mixing element 140 is provided within the reservoir to agitate the organic material and promote uniform heating during decarboxylation and infusion protocols. A memory 150 stores operational settings for the apparatus controller 100 for various organic materials and infused solvent products that can be created. Each operational setting is selectable using a user interface 160 provided on the apparatus 100 or a computing device 170 in communication with the user. Network 180 transmits and receives data to and from the computing device 170 and database 190 to the apparatus controller 100.

In some embodiments, the alert system 120 emits an alert corresponding to the operational status of the apparatus during use. For example, the alert system 120 may be in operable communication with a speaker(s) and/or light(s) corresponding to an operation status (e.g., when the heating process is done, when the cooling process is done, when decarboxylation is complete, when infusion is complete, etc.).

In some embodiments, instructions for operational settings are stored in the database, which can include hardware components or cloud-based data storage. The computing device displays selectable options to the user, which are transmitted via the network to operate the apparatus.

In some embodiments, the alert system alerts the user using the apparatus and/or the computing device upon completion of the decarboxylation and infusion protocols. Alerts can include any audio or visual means known in the arts.

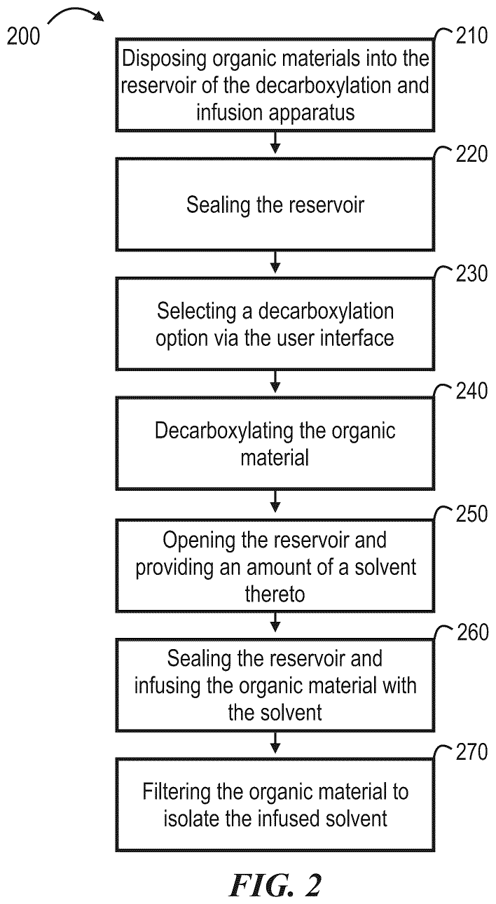

FIG. 2 illustrates a method of use 200 of the apparatus. In step 210, the user disposed of organic materials into the reservoir of the decarboxylation and infusion apparatus 100. In step 220, the reservoir is sealed, and the user selects a suitable decarboxylation protocol using a user interface in step 230. The decarboxylation protocol may be altered depending on the organic material used and molecule to be decarboxylated. Following the decarboxylation protocol in step 240, the reservoir is opened, and a solvent is provided in step 250. In step 260, the reservoir is sealed, and the organic material is infused with the solvent to produce an infused solvent product. In step 270, the organic material is filtered to isolate the infused solvent, which can then be added to a foodstuff, beverage, topical, or another delivery mechanism.

In some embodiments, the user may select for isolation of one or more molecules by selecting an infusion pressure, an infusion temperature, one or more solvents, and infusion time. For example, the user can select to infuse CBD into the solvent without THC to reduce the psychoactive effects of the infused solvent.

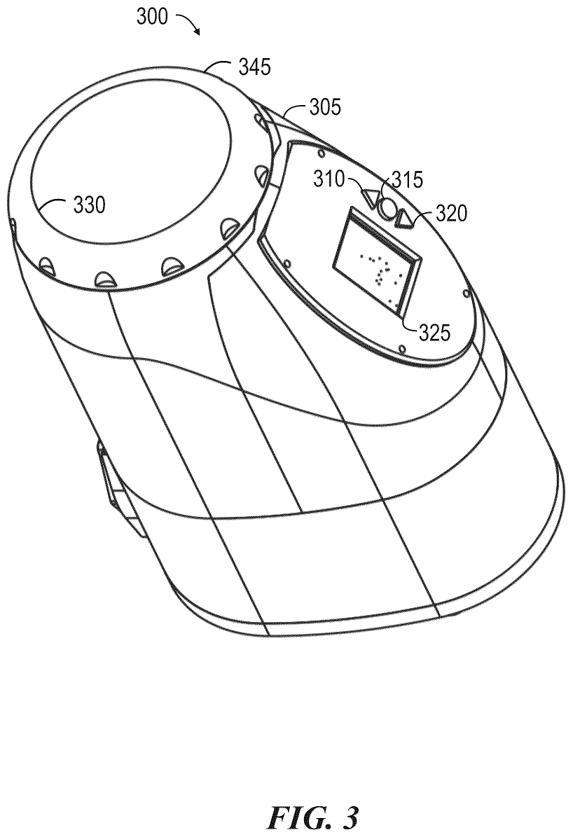

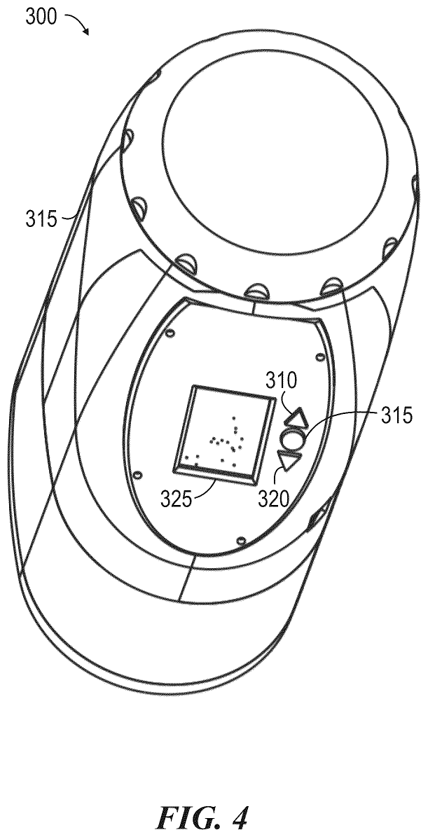

FIG. 3 and FIG. 4 illustrate the decarboxylation and infusion apparatus 300 comprising a housing 305 having controls 310, 315, 320 (collectively referred to as “controls”), and interface 325. Each of the controls permits the user to interact with the interface 325 to select various functionalities and operational parameters of the decarboxylation and infusion apparatus 300 to effectively decarboxylate and/or infuse or otherwise prepare the organic material.

In some embodiments, the interface 325 is configured as a touchscreen interface which provides the user the ability to control the various functionalities of the apparatus. For example, the user may select various heat settings, cooling settings, and/or timing settings. Further, the memory may include various pre-programmed settings for various types of organic material, various amounts of each type of organic material, or intended final product which are decarboxylated and infused by the apparatus.

In further reference to FIG. 3, a lid 330 is provided at the top portion 345 of the apparatus 300. The lid 330 may include a safety system which includes a sensor which determines if the lid 330 is in an open or closed position. When the lid 330 is in a closed configuration, the apparatus may perform the various tasks of decarboxylating the organic material. For example, when the lid 330 is in a closed configuration, the apparatus may perform the various functions necessary to decarboxylate organic material, to blend organic material, and/or to infuse the organic material.

In some embodiments, the sensor, when sensing an open position, may instruct the processor prevent specific functions of the apparatus once the lid 330 is in the open position. For example, if the lid 330 is open, the apparatus may be unable to blend the organic material, heat the organic material, or otherwise decarboxylate and infuse the organic material. In such, the sensor provides a safety mechanism to prevent injury or other unexpected and unintended results.

In some embodiments, the interface 325 is in operable communication with a speaker to provide audio feedback to the user. The speaker may emit alerts to the user once various processes are starting, in progress, and/or completed. For example, the speaker may emit a sound once the decarboxylation and/or infusion processes are completed.

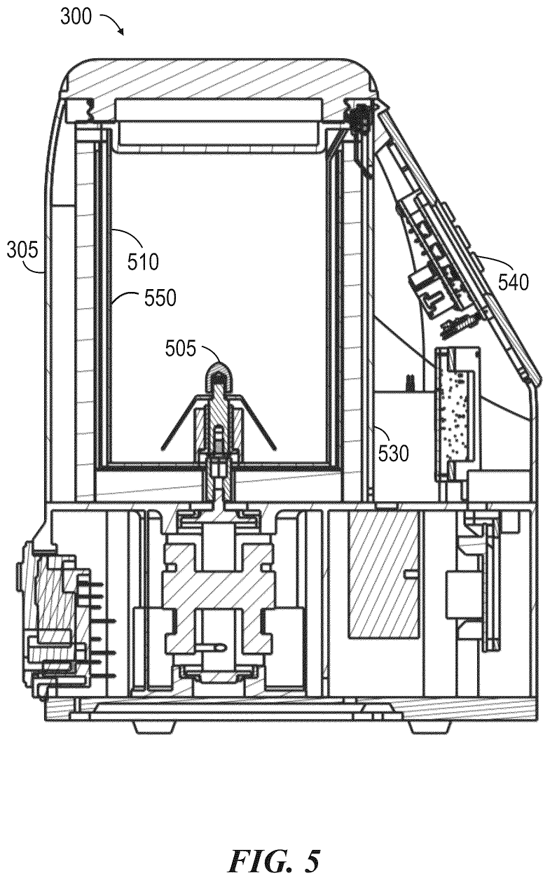

FIG. 5 illustrates a cross-section view of the decarboxylation and infusion apparatus 300 to show the housing 305 and the internal components thereof. A mixing element 505 is provided within a heated reservoir 510 to agitate organic material and a solvent disposed of therein. One skilled in the arts will readily understand that the organic material and solvent may change depending on the application of the product. A fan 530 is positioned within the housing 305 to aid in heat dissipation following the decarboxylation and infusion processes. The heat dispersion may aid in maintaining the quality by preventing degradation of the final product. Further, the fan 530 may aid in the removal and/or the redirection of odors produced throughout the process. The fan 530 is in communication with a vent 540 to permit the egress of air.

In some embodiments, the fan 530 is in operable communication with the controller 100 to provide operational controls to the fan 530 during cooling and deodorization. In such, the user may select a fan speed, deodorization settings, fan timing, and other controls to suitably cool the mixing chamber and deodorize air emitted from the vent 540.

In some embodiments, the vent 540 may include a filter such as carbon (charcoal) filtration, or similar filtration means to remove odors from the expelled air. The vent 540 may include one or more air expulsion elements, including auxiliary fans, to direct air from the mixing chamber through the vent 540 wherein the odor is neutralized, removed, or masked to prevent the odors within the mixing chamber from being emitted into the surrounding environment.

In some embodiments, the auxiliary fans allow expel heat via the vents 540 which allow the contents within the chamber to rapidly cool. This increases the quality of the final product by providing an apparatus which has precise and efficient heating and cooling systems.

In some embodiments, the heated reservoir is at least partially surrounded or encapsulated by an insulative layer 550 to thermally isolate the heated reservoir and the product therein within the apparatus from the environment and its ambient heating conditions as well as to evenly distribute the heat. An airtight lid may be provided to prevent oxygen from entering mixing chamber during the decarboxylation and infusion processes, minimize evaporation, and reduce odors.

In some embodiments, the insulative layer 550 prevents the material from being in contact with direct heat from the heating element. In such, the insulative layer 550 protects the material from degradation due to excessive heat.

In some embodiments, the heated reservoir 510 includes a spout to facilitate pouring of the product created from the decarboxylation and infusion process. The spout may also prevent counterrotation during blending. The spout may be configured as a complimentary shape to the housing the apparatus to prevent counterrotation and to ensure the heated reservoir 510 is properly positioned during the blending, decarboxylation and infusion processes.

The housing may be constructed of a variety of materials which are suitable, including, but not limited to, plastic, glass, silicone, food-grade butyl rubber, latex, aliphatic polyesters, natural rubber, metal, metal foils, polytetrafluoroethylene, biopolymers such as liquid wood, modified casein, polyhydroxyalkanoate polyesters, including polyhydroxybutrate, polyhydroxyvalerate, polylactic acid, starch-based polyesters, keratin processed with methyl acrylate, hemp polymers, hemp plastic, hemp composite polymers, and combinations thereof.

In some embodiments, the decarboxylation and infusion apparatus described herein provides a means for a semi-automated system for performing the chemical processes of decarboxylating an organic material and infusing the decarboxylated organic material into a solvent for various applications, including consumption by a human. The apparatus may be provided as a single contained unit within a housing to prevent contamination, or undue transfer of the raw or processes materials.

The housing may be provided in various configurations to provide a single containerized heated reservoir and mixing chamber and mixing element to decarboxylate an organic material and infuse the organic material with a solvent. The infused solvent may then be extracted from the housing and packaged or otherwise processed into a final product.

The preferable heat-cooking steps may comprise only one dry heat cooking step, multiple dry heat-cooking steps, and/or dry heat-cooking step(s) with blended herbs, chemicals, and flavorings.

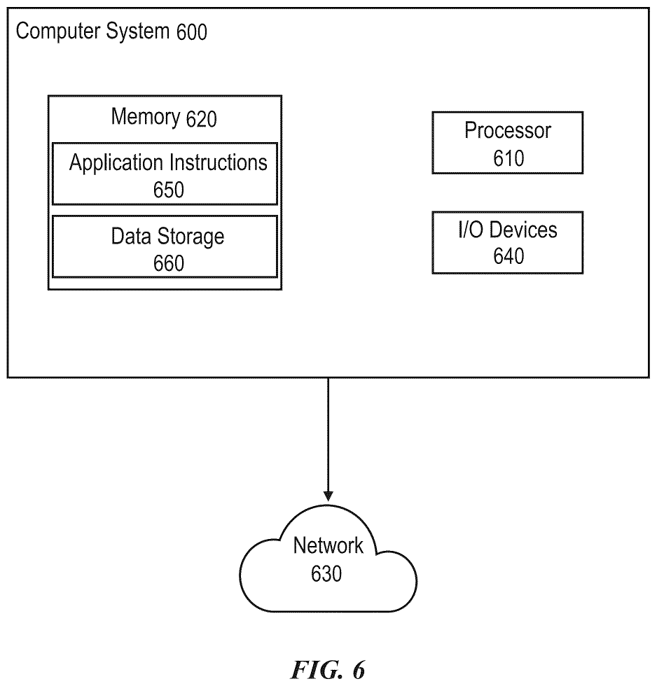

FIG. 6 illustrates a block diagram of the network infrastructure which may be used to control the decarboxylation and infusion apparatus which may be controlled via the touchscreen interface, and/or a mobile device, computer, and the like. The computer system 600, which may be utilized to execute the processes described herein. The computer system 600 is comprised of a standalone computer or mobile computing device, a mainframe computer system, a workstation, a network computer, a desktop computer, a laptop, or the like. The computer system 600 includes one or more processors 610 coupled to a memory 620 via an input/output (I/O) interface. Computer system 600 may further include a network interface to communicate with the network 630. One or more input/output (I/O) devices 640, such as video device(s) (e.g., a camera), audio device(s), and display(s) are in operable communication with the computer system 600. In some embodiments, similar I/O devices 640 may be separate from computer system 600 and may interact with one or more nodes of the computer system 600 through a wired or wireless connection, such as over a network interface.

Processors 610 suitable for the execution of a computer program include both general and special purpose microprocessors and any one or more processors of any digital computing device. The processor 610 will receive instructions and data from a read-only memory or a random-access memory or both. The essential elements of a computing device are a processor for performing actions in accordance with instructions and one or more memory devices for storing instructions and data. Generally, a computing device will also include, or be operatively coupled to receive data from or transfer data to, or both, one or more mass storage devices for storing data, e.g., magnetic, magneto-optical disks, or optical disks; however, a computing device need not have such devices. Moreover, a computing device can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

A network interface may be configured to allow data to be exchanged between the computer system 600 and other devices attached to a network 630, such as other computer systems, or between nodes of the computer system 600. In various embodiments, the network interface may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol.

The memory 620 may include application instructions 650, configured to implement certain embodiments described herein, and a database 660, comprising various data accessible by the application instructions 650. In one embodiment, the application instructions 650 may include software elements corresponding to one or more of the various embodiments described herein. For example, application instructions 150 may be implemented in various embodiments using any desired programming language, scripting language, or combination of programming languages and/or scripting languages (e.g., C, C++, C#, JAVA®, JAVASCRIPT®, PERL®, etc.).

The steps and actions of the computer system 600 described in connection with the embodiments disclosed herein may be embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 610 such that the processor 610 can read information from, and write information to, the storage medium. In the alternative, the storage medium may be integrated into the processor 610. Further, in some embodiments, the processor 610 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

Also, any connection may be associated with a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. “Disk” and “disc,” as used herein, include compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

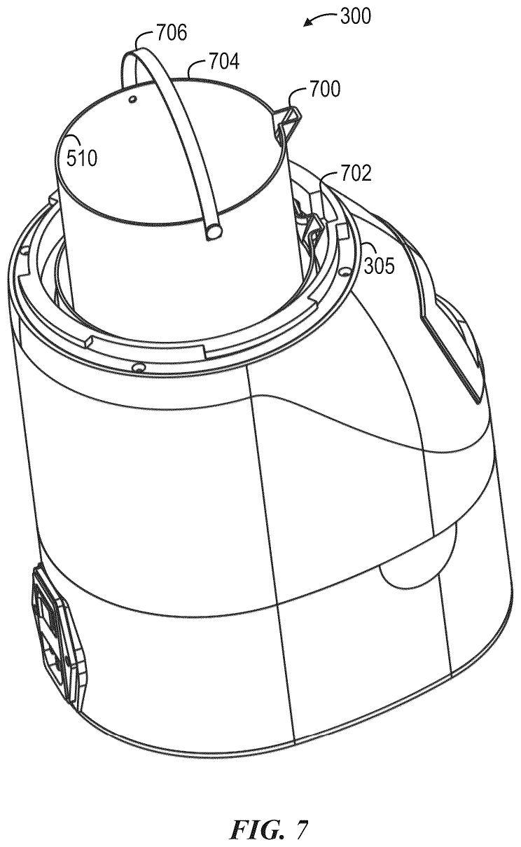

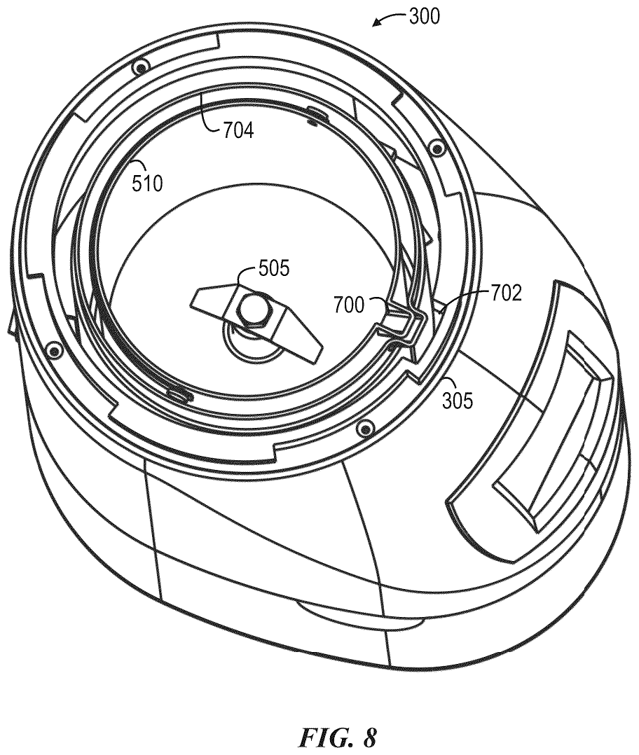

FIG. 7 and FIG. 8 illustrate a perspective view of the decarboxylation and infusion apparatus 300 wherein the heated reservoir 510 is removable from the apparatus. The heated reservoir 510 includes a spout 700 which fittingly engages with a complimentary portion 702 molded into the housing 305 to prevent counterrotation during blending of the organic material. The spout 700 is positioned at the perimeter 704 of the heated reservoir 510. The handle 706 may be provided on the heated reservoir 510 to facilitate the removal of the heated reservoir 510 from the housing 305. In specific reference to FIG. 8, the mixing element 505 is shown. During operation, the mixing element 505 may emit a force onto the heated reservoir 510 causing a counterrotation. This counterrotation is stopped by the spout 700 fittingly engaging with the complimentary portion 702 of the housing 305.

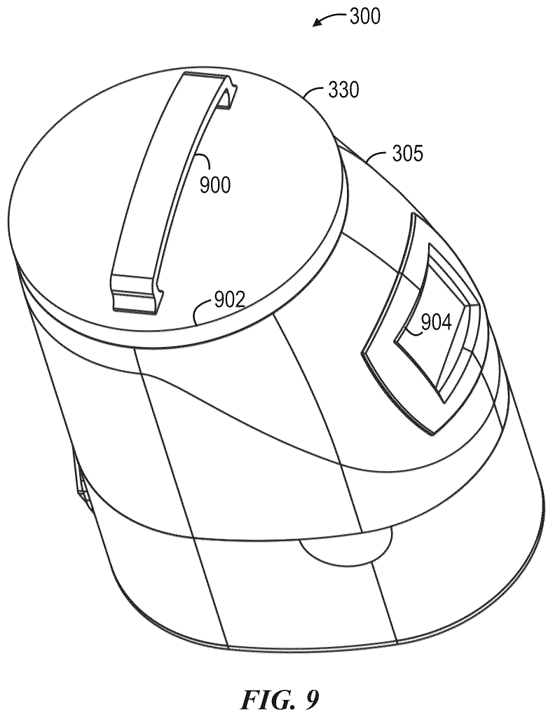

FIG. 9 illustrates a perspective view of the decarboxylation and infusion apparatus 300 having an airtight lid 330 which is engaged to the housing 305. A handle 900 is positioned on a top surface 902 of the lid 330 which allows the user to easily pick up and carry the apparatus 300 as well as facilitates the removal of the lid 330. This lid 330 may include a locking mechanism which is activated to ensure the lid 330 is not opened during the mixing, decarboxylation, and/or infusion processes.

In some embodiments, the apparatus may utilize a touchscreen interface 904 rather than buttons (as shown in FIGS. 3-5 310, 315, 320) to allow the user to select operation settings.

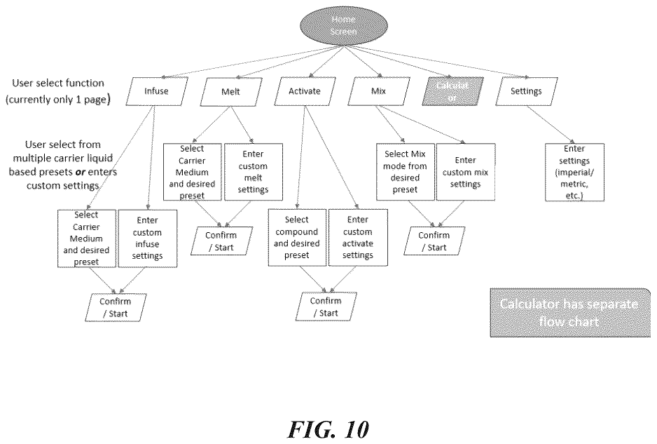

Referring now to FIG. 10, a flowchart is provided illustrating an exemplary embodiment of the infusion calculation module process executed by the system. The process begins at a home screen interface presented to the user either on the apparatus's touchscreen or on a mobile device application in wireless communication with the apparatus. From the home screen, the user is presented with several primary function options, including “Infuse,” “Melt,” “Activate,” “Mix,” “Calculator,” and “Settings.” Each of these modules corresponds to a specific category of operation available through the infusion system.

The home screen interface may be a single screen or multiple screens which enables the user to select form various operational functions of the system.

In some embodiments, the system leverages smart technology to both suggest and enact optimal processing parameters, such as infusion temperature, mixing speed, melting duration, or cooling ramp profiles, based on the specific ingredient-carrier combination selected by the user. Upon initiation of a workflow, the user may input or select the active ingredient and the carrier solvent (e.g., shea butter, olive oil, glycerin, etc.), after which the system queries a compound-aware logic engine housed within the ELOH database. This logic engine includes pre-populated and continuously updatable datasets describing thermal degradation thresholds, solubility characteristics, mixing tolerances, and infusion profiles for a wide range of compounds. Based on this data, the system automatically recommends a time-temperature-mixing curve optimized for the selected ingredients. The user may accept, modify, or override these settings; however, in default or guided mode, the system autonomously controls the hardware to carry out the entire cycle with minimal intervention. The system may also adapt these suggestions in real time based on live sensor input, such as temperature or viscosity changes, to ensure the process remains within safe and effective ranges. This smart decision-making framework enables consistent, high-quality outcomes across diverse formulation types while reducing the need for user expertise or trial-and-error.

In reference to FIG. 10, the potency estimation software operates as a standalone calculator module as well as an integrated component of the guided infusion workflow. Upon initiating an infusion session from the main interface, whether via the device's touchscreen or a paired mobile application, the user may select the “Calc” or “Calculator” icon, which launches the potency estimation sequence. The system prompts the user to input key variables such as the weight of the source material, estimated compound concentration (e.g., percentage of active ingredient), total carrier volume, and desired serving size. These values are passed to the potency estimation engine, which applies stored extraction efficiency coefficients and degradation factors to compute both total and per-serving potency. The interface presents results in mg/mL, mg per dose, and percentage by weight, along with a summary of the assumptions used in the calculation.

When the user selects the “Infuse” function, the system presents a submenu that allows the user to select a carrier medium from a list of available solvents or oils. These carrier mediums may include, but are not limited to, olive oil, coconut oil, butter, waxes, or glycerin. At this stage, the user is presented with two options: either selecting a predefined preset infusion profile associated with the chosen carrier medium, or manually entering custom infusion parameters. Presets are pre-configured profiles stored in the system's memory and may include optimal heating times, temperature ranges, and agitation cycles developed for specific combinations of material and solvent.

If the user chooses to use a preset infusion setting, the system will automatically populate the corresponding thermal and mechanical settings and display a confirmation screen. The user may review the summary of selected parameters and initiate the infusion cycle by selecting the “Confirm/Start” prompt. This step triggers the main controller to begin executing the infusion routine based on the parameters provided. The system then initiates pre-heating of the chamber, followed by staged mixing and infusion of the organic material within the selected carrier medium.

Alternatively, if the user prefers to input custom infusion settings, the system presents a data entry interface that allows for manual specification of temperature setpoints, infusion durations, agitation intervals, and cooling options. This flexibility enables experienced users to tailor the infusion process to novel materials, proprietary formulations, or desired potency outcomes. Once all parameters are entered, the system again presents a confirmation screen. Upon user confirmation, the system saves the custom profile (if applicable) and proceeds with execution of the infusion cycle.

The “Melt” function operates in a similar fashion. The user is prompted to either select a carrier medium and associated preset, or to enter custom melt settings manually. The melt function may be used to prepare a base solvent prior to addition of the organic material, or to soften ingredients before full blending. After parameter selection, the user confirms and initiates the melt cycle.

The “Activate” function corresponds to the decarboxylation of the organic material and is accessed in parallel with the other modules. Upon selecting the “Activate” function, the user selects the organic compound type or strain and chooses from available presets or manually enters a decarboxylation protocol. These parameters may include low-temperature baking for a prolonged duration or high-temperature flash activation. After input or selection, the user is guided to a confirmation step and proceeds to activation by initiating the process through the interface.

The “Mix” function allows the user to configure the system to blend or emulsify materials without applying heat. From the Mix submenu, the user can select from a list of preset mixing profiles or enter custom parameters. Presets may include low-speed emulsification, high-speed blending, or intermittent mixing protocols. This function may be used during pre-infusion preparation or post-infusion homogenization. Following parameter configuration, the user confirms the input and activates the mixing process.

The “Calculator” function is a standalone module configured to estimate final infusion potency based on user-provided inputs. The calculator may operate independently of the active infusion cycles and is accessed directly from the home screen. Once launched, the user is prompted to enter variables such as material weight, compound type, solvent volume, and process time and temperature. The system uses these inputs to compute estimated potency using preloaded formulas, tables, or machine-learned algorithms. The potency calculator includes its own flowchart and output sequence, which may present results in various formats such as mg/mL, mg per dose, or percentage by weight.

The “Settings” function provides system-level configuration and customization. This includes toggling between imperial and metric units, adjusting audio or visual feedback preferences, updating software, and managing user profiles or permissions. Settings may also include calibration options for weight sensors, cleaning cycle preferences, and connectivity management. By providing these tools, the system accommodates a wide range of user needs and operational contexts.

Overall, the flowchart illustrated in FIG. 10 provides a flexible and modular framework for infusion management. Whether the user chooses a fully guided preset routine or inputs customized parameters for each phase, the system supports a structured and user-friendly approach to complex infusion tasks. The integration of the infusion calculator, presets, custom profile entry, and mobile interface ensures that users at every experience level can reliably produce high-quality infused products with minimal effort or error.

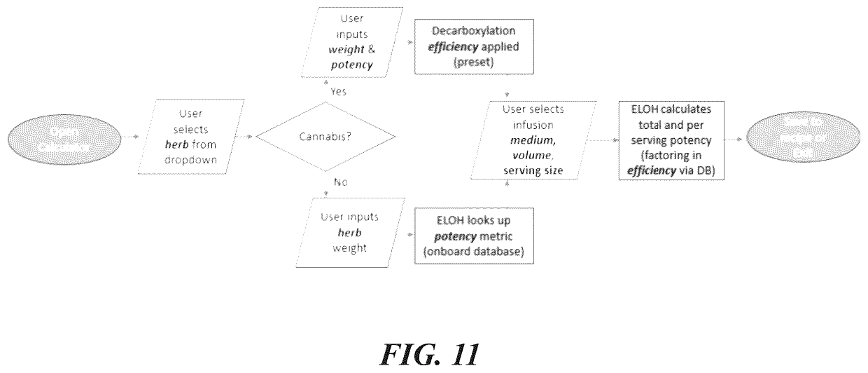

Referring now to FIG. 11, a flowchart is shown which illustrates an embodiment of the infusion potency calculator process. This process is executed by the infusion system's embedded software or mobile application and is configured to calculate both total and per-serving potency of an infused material. The calculator function may be accessed via the system's touchscreen interface or a wirelessly connected mobile device. Upon activation of the calculator module, the user is guided through a series of inputs and decision branches to obtain an accurate potency estimate.

As shown in FIG. 11, the potency estimation flowchart illustrates the internal decision-making logic used to generate accurate potency predictions. The software begins by verifying the compound class and material type (e.g., dry herb, extract, powdered root, concentrate), then queries the ELOH database for the corresponding infusion efficiency and compound yield profile. In cases where decarboxylation or thermal activation is not relevant, the system skips the conversion efficiency step and instead applies a direct absorption or solubility coefficient. For each input, the system may apply confidence intervals or error margins based on user-supplied variables such as material freshness, storage conditions, or batch variability. Once all calculations are complete, the system generates a report that includes estimated final compound concentration, carrier saturation level, and suggested serving measurements.

The first step in the process prompts the user to select an herbal material from a dropdown list. This list may include herbs, roots, flowers, or other botanical substrates designated by form (e.g., fresh, dry, powdered). In some embodiments, the list includes cannabis and non-cannabis materials, each tagged with metadata including common preparation methods, average potency metrics, and recommended infusion mediums. Once the user selects an herb, the system queries whether the selected material is cannabis or another plant-based substance.

If the selected herb is cannabis, the system presents an input form requesting both the weight of the cannabis material and its known or disclosed potency. Potency in this context may refer to a known concentration of a key compound, such as THC or CBD, expressed as a percentage of total dry weight. The user may input weight in grams or ounces, and the system may automatically convert units to maintain internal consistency. In some embodiments, the cannabis potency may be scanned or imported from a certificate of analysis or product label using optical recognition or barcode scanning.

Upon receiving the weight and potency input, the system applies a decarboxylation efficiency factor to account for activation loss during the heating process. This efficiency factor may be preset or user-configurable and reflects expected degradation or conversion inefficiencies inherent to the decarboxylation process. For instance, if the user inputs 1 gram of cannabis with 20% THC, and the system applies a 75% decarboxylation efficiency, the system calculates 150 mg of activated THC as the available input compound. This ensures that potency predictions reflect actual molecular bioavailability post-processing.

In contrast, if the selected herb is a non-cannabis plant (e.g., turmeric, chamomile, or echinacea), the user is prompted to input the weight of the material. The system then looks up a corresponding potency metric using an onboard database. This database, referred to as the Embedded Lookup of Herbal Outputs (ELOH), contains empirically derived or literature-supported potency values for common bioactive compounds found in various botanicals. These values may be expressed per gram, ounce, or other standardized measures and may include compound names such as curcumin, flavonoids, alkaloids, or terpenes.

Once the system has established a potency baseline, whether through user input or ELOH lookup, it prompts the user to select additional infusion parameters. These parameters include the infusion medium (e.g., oil, butter, glycerin, syrup), total volume of the solvent, and the desired serving size. The volume may be input in milliliters or ounces, while the serving size may be selected from a dropdown menu of common measures (e.g., teaspoon, tablespoon, 1 mL) or entered manually. This enables the system to map the total infused compound over the intended number of servings.

The calculator module integrates all of the collected data to compute the total expected potency of the final infusion and the per-serving potency. These calculations account for decarboxylation efficiency (if applicable), infusion efficiency (which reflects losses during straining, evaporation, or incomplete transfer), and the relative concentration of compound per unit of volume. For example, if 150 mg of active compound is infused into 100 mL of oil and the serving size is 5 mL, the system will report 7.5 mg per serving.

In some embodiments, the calculator module accounts for transformative agents (e.g., citric acid), to increase scent potency or (e.g., piperine) to increase curcumin bioavailability.

Infusion efficiency is an internal metric derived from prior data or user calibration and may vary depending on the herb, solvent, temperature, and processing duration. This value may be fixed for general use or adjusted based on material freshness, moisture content, or user feedback. Efficiency values are typically less than 100% to account for unavoidable losses. The system may allow users to manually override this value to better reflect observed yields in specialized cases.

After the system performs the calculation, it presents the results to the user via the interface. This may include a summary report showing total potency, number of servings, potency per serving, and assumptions used in the computation. In some embodiments, the system displays graphical output such as pie charts, bar graphs, or potency meters to visually communicate results. The user may then choose to save the calculated infusion recipe or exit the calculator.

If the user selects “Save to Recipe,” the system stores the current parameters in a user-accessible library of infusion profiles. These saved recipes may be recalled and executed by the system in future sessions. Saved entries include all relevant variables such as herb type, weight, solvent, temperature targets, and serving size. The ability to save, recall, and refine infusion recipes provides continuity and supports consistency across repeated use.

Overall, the calculator module illustrated in FIG. 11 offers a structured and data-informed method for predicting infusion potency across a wide variety of botanical materials and carrier solvents. By integrating compound-specific efficiency factors, serving size calculations, and an onboard database of potency metrics, the system provides an enhanced level of accuracy and user empowerment. The calculator module may be implemented as a standalone tool or integrated seamlessly into the broader guided infusion system.

In some embodiments, the calculator module may utilize data-informed predictions for diverse botanicals. The calculator module may allow for user guidance by using known amounts of compounds and carriers which can better inform processing times.

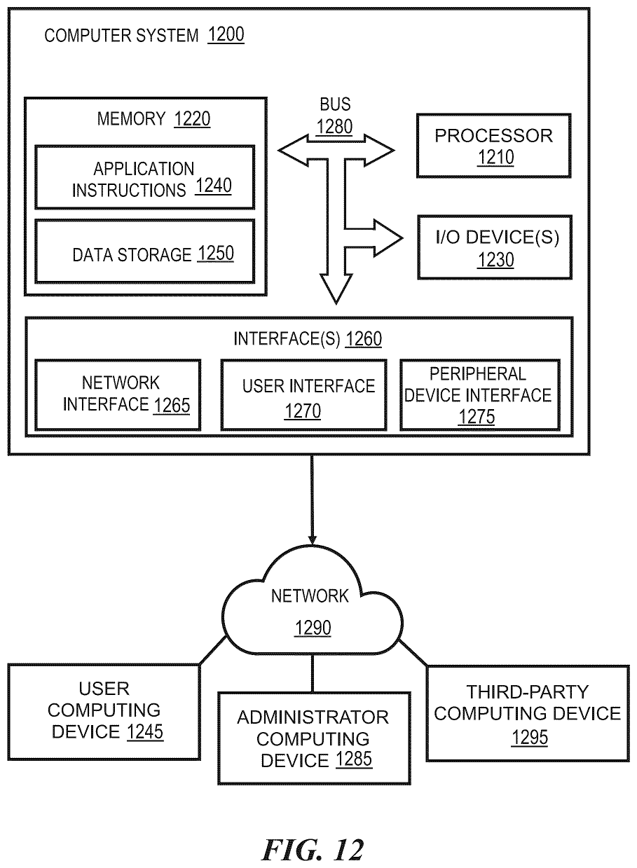

FIG. 12 illustrates an example of a computer system 1200 that may be utilized to execute various procedures, including the processes described herein. The computer system 1200 comprises a standalone computer or mobile computing device, a mainframe computer system, a workstation, a network computer, a desktop computer, a laptop, or the like. The computing device 1200 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 1200 of FIG. 12 may be used for any infusion target described herein including syrups, balms, etc. The sensors which are in operable communication with the computer system may be used to triangulate temperatures needed for specific processing of temperature requirements.

In some embodiments, the computer system 1200 includes one or more processors 1210 coupled to a memory 1220 through a system bus 1280 that couples various system components, such as an input/output (I/O) devices 1230, to the processors 1210. The bus 1280 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 1200 includes one or more input/output (I/O) devices 130, such as video device(s) (e.g., a camera), audio device(s), and display(s) are in operable communication with the computer system 1200. In some embodiments, similar I/O devices 130 may be separate from the computer system 1200 and may interact with one or more nodes of the computer system 100 through a wired or wireless connection, such as over a network interface.

Processors 1210 suitable for the execution of computer readable program instructions include both general and special purpose microprocessors and any one or more processors of any digital computing device. For example, each processor 1210 may be a single processing unit or a number of processing units and may include single or multiple computing units or multiple processing cores. The processor(s) 1210 can be implemented as one or more microprocessors, microcomputers, microcontrollers, digital signal processors, central processing units, state machines, logic circuitries, and/or any devices that manipulate signals based on operational instructions. For example, the processor(s) 1210 may be one or more hardware processors and/or logic circuits of any suitable type specifically programmed or configured to execute the algorithms and processes described herein. The processor(s) 1210 can be configured to fetch and execute computer readable program instructions stored in the computer-readable media, which can program the processor(s) 1210 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 1220 includes computer-readable application instructions 150, configured to implement certain embodiments described herein, and a database 1250, comprising various data accessible by the application instructions 1240. In some embodiments, the application instructions 1240 include software elements corresponding to one or more of the various embodiments described herein. For example, application instructions 140 may be implemented in various embodiments using any desired programming language, scripting language, or combination of programming and/or scripting languages (e.g., Android, C, C++, C#, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 1240 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 1210 such that the processor 1210 can read information from, and write information to, the storage medium. In the alternative, the storage medium may be integrated into the processor 1210. Further, in some embodiments, the processor 1210 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 1240 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 1240 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 1240 can be downloaded to a computing/processing device from a computer readable storage medium, or to an external computer or external storage device via a network 1290. A network adapter card or network interface in each computing/processing device receives computer readable program instructions from the network and forwards the computer readable application instructions 1240 for storage in a computer readable storage medium within the respective computing/processing device.

In some embodiments, the computer system 1200 includes one or more interfaces 160 that allow the computer system 1200 to interact with other systems, devices, or computing environments. In some embodiments, the computer system 1200 comprises a network interface 1265 to communicate with a network 1290. In some embodiments, the network interface 1265 is configured to allow data to be exchanged between the computer system 1200 and other devices attached to the network 1290, such as other computer systems, or between nodes of the computer system 1200. In various embodiments, the network interface 1265 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 1270 and the peripheral device interface 1275.

In some embodiments, the network 1290 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 1290 can comprise copper transmission cables, optical transmission fibers, wireless transmission, routers, firewalls, switches, gateway computers and/or edge servers. The network 1290 can represent a single network or multiple networks. In some embodiments, the network 1290 used by the various devices of the computer system 1200 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 1200 may include a user computing device 145, an administrator computing device 1285 and a third-party computing device 1295 each in communication via the network 1290. The user computing device 1245 may be utilized a user (e.g., a healthcare provider) to interact with the various functionalities of the system including to perform patient rounds, handoff patient rounding responsibility, perform biometric verification tasks, and other associated tasks and functionalities of the system. The administrator computing device 1285 is utilized by an administrative user to moderate content and to perform other administrative functions. The third-party computing device 1295 may be utilized by third parties to receive communications from the user computing device, transmit communications to the user via the network, and otherwise interact with the various functionalities of the system.

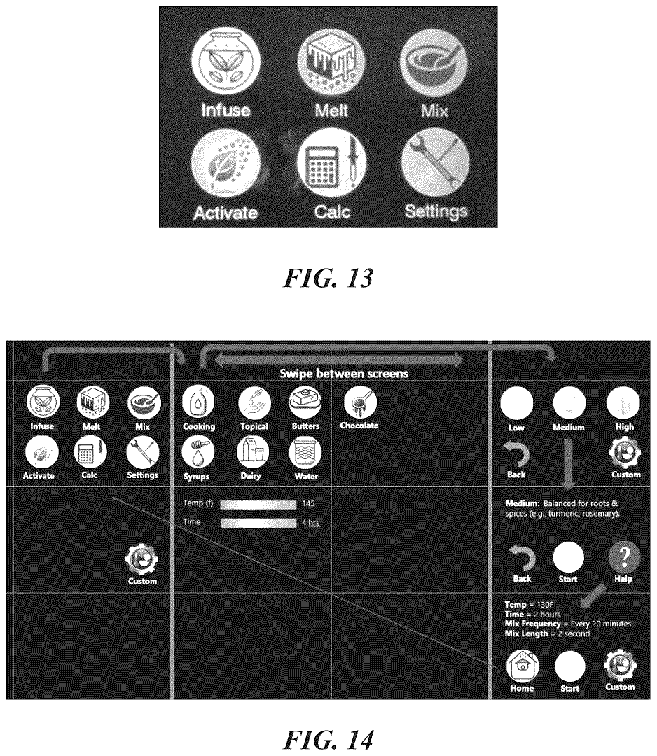

Referring now to FIG. 13, a screenshot is shown depicting the user interface of the application program, which is presented on a touchscreen display integrated into the apparatus or accessed via a connected mobile device. The interface measures approximately three inches in width and two inches in height and presents six selectable icons arranged in two rows. Each icon corresponds to a core functional module of the infusion system, allowing the user to intuitively navigate between operational modes including “Infuse,” “Melt,” “Mix,” “Activate,” “Calc,” and “Settings. ” The graphical design and layout of the user interface are optimized for clarity, ease of use, and responsiveness, ensuring efficient interaction regardless of user experience level.

The user interface elements depicted in FIG. 13 include a dedicated icon for accessing the potency calculator, labeled “Calc.” When selected, this module overlays an input form onto the main display, prompting the user to enter ingredient weight, compound concentration, carrier volume, and optionally, bioavailability enhancers or transformative agents. The system is capable of adapting its behavior based on whether the active compound is thermal-sensitive, fat-soluble, water-soluble, or unstable in certain carrier environments. For example, when a user selects a water-based infusion and inputs a compound known to have poor aqueous solubility, the system may issue a warning or recommend an alternative carrier. These smart validations are enabled by dynamic reference to the ELOH database, which stores solubility tables, degradation thresholds, and compound-carrier compatibility profiles.

The “Infuse” option initiates the infusion protocol, allowing the user to either select a preset infusion profile or input custom temperature, time, and agitation settings. The “Melt” option is used for preparing or softening carrier mediums such as butter, wax, or oils before combining them with the active material. The “Mix” function operates the integrated mixing mechanism independently of heating, enabling pre-or post-infusion homogenization of contents. The “Activate” module manages decarboxylation routines for activating compounds in raw organic material through controlled heating cycles. The “Calc” (calculator) icon launches the infusion potency estimation module, which computes both total and per-serving compound concentrations based on user inputs and onboard efficiency metrics. Lastly, the “Settings” option provides access to system preferences including measurement units, interface language, sound alerts, cleaning routines, and software updates. These modular functions collectively support a flexible and intelligent infusion process tailored to user needs.

Referring now to FIG. 14, a schematic illustration is provided which depicts the multi-screen user interface functionality of the smart infusion system. The diagram highlights the system's ability to support an intuitive and visually guided user experience through swipeable display screens, each tailored to specific functions and ingredient categories. This modular, graphical user interface enables a user to navigate between infusion options, carrier mediums, and mixing profiles with minimal complexity, providing a streamlined operational environment suitable for both novice and advanced users. The layout presented in FIG. 14 is exemplary of a touchscreen workflow adaptable for use on the device itself or through a companion mobile application.

Referring now to FIG. 14, the potency estimation software integrates with the multi-screen navigation interface to allow seamless inclusion of potency guidance throughout the infusion workflow. As the user swipes through the infusion setup screens, the system may dynamically update estimated potency values in real time based on the current input selections. For example, if the user increases the carrier volume from 100 mL to 200 mL, the system will immediately recalculate the per-serving concentration and display updated values in the lower portion of the interface. This real-time feedback loop ensures that users understand how changes in input parameters affect the final outcome and supports iterative refinement prior to starting the infusion cycle.

The first row of the interface includes the primary function options: “Infuse,” “Melt,” “Mix,” “Activate,” “Calc,” and “Settings.” These icons, previously described in reference to FIG. 13, form the main menu and serve as gateways into more specialized operational workflows. Swiping left or right across the screen transitions the user between different stages or categories, allowing access to secondary menus without exiting the core program. This continuity of navigation minimizes friction during setup and enables the user to build a comprehensive infusion recipe in a sequential manner.

The second screen, accessed by swiping from the main menu, displays a set of application-specific infusion targets such as “Cooking,” “Topical,” “Butters,” “Chocolate,” “Syrups,” “Dairy,” and “Water.” Each icon represents a common use-case or solvent category, allowing the user to select a carrier type relevant to the intended application of the infused product. For example, selecting “Topical” may prompt different heating and mixing parameters than selecting “Cooking” or “Chocolate,” as different viscosities and volatility levels require tailored thermal profiles. These presets are linked to stored infusion profiles that adjust the apparatus's temperature and timing settings accordingly.

Beneath these functional icons, the interface provides real-time status indicators for temperature and infusion time. For instance, the display may indicate that the target temperature is 145° F. and that the process will run for 4 hours. These readouts help the user visually verify and confirm the operational parameters before and during the process. In some implementations, the system may dynamically adjust these parameters based on sensor feedback or infusion progress, and the changes will be reflected in real time on this screen.

Additionally, the interface supports a tiered selection model for mixing intensity. The user can select from “Low,” “Medium,” or “High” mixing profiles, each corresponding to a different frequency and duration of the mixing cycle. For example, a “Medium” setting may be ideal for roots and spices like turmeric or rosemary, delivering balanced agitation over a defined time interval. Upon selection of a mixing profile, a summary of the associated values (e.g., mix every 20 minutes for 2 seconds) is displayed, providing transparency and allowing the user to fine-tune the profile as needed.

For more advanced control, a “Custom” option is available at multiple stages of the workflow. This option allows the user to input or modify parameters including infusion temperature, total run time, mix frequency, and mix duration. These values can be entered manually using slider controls, numeric keypads, or dropdowns, depending on the implementation. This flexibility is particularly useful for users who wish to experiment with new materials or develop proprietary infusion protocols. Selecting “Custom” overrides the default values linked to the pre-configured icons.

The lower right portion of the interface includes core navigational tools such as “Back,” “Start,” and “Help.” These buttons provide immediate access to critical functions, ensuring that the user can navigate the interface without confusion. For example, the “Help” button may offer contextual information or tooltips based on the screen currently in view, assisting the user in understanding infusion science, safety protocols, or interface options. The “Start” button, when selected, initiates the infusion process based on the chosen parameters and transitions the device into an active processing mode.

The final row of the schematic shows detailed process metrics such as current temperature, time remaining, mix frequency, and mix length. For example, it may indicate a setting of 130° F. for 2 hours, with mixing every 20 minutes for a duration of 2 seconds. These data points are critical for achieving consistency in infused product outcomes and may be saved as part of a custom recipe for future use. A “Home” icon enables quick return to the main screen from any point within the interface hierarchy, reinforcing the system's emphasis on user-friendly design.

In sum, FIG. 14 illustrates a responsive, swipe-based interface that integrates all core operational elements of the smart infusion system into a cohesive and ergonomic control environment. The layered menu system, preset application categories, dynamic parameter displays, and advanced customization options work in concert to deliver an interactive user experience tailored to both ease of use and functional depth. The interface architecture supports the broader system goals of accuracy, flexibility, and process transparency, enabling consistent preparation of high-quality infused compositions.

In one embodiment, the system is configured to perform infusion of botanical materials into various carrier liquids using selectable temperature, time, and agitation profiles. As shown in the “Infuse Settings” data, the system offers pre-programmed infusion levels, Low, Medium, and High, each corresponding to specific time and temperature ranges based on the selected carrier medium. For example, cooking oils may be infused under low settings at 120° F. for 4 hours, medium at 130° F. for 2 hours, or high at 140° F. for 1 hour. The user may select these presets based on the delicacy or toughness of the plant material to ensure optimal extraction without degradation.

In another embodiment, the infusion system includes tailored topical oil profiles. These are designed for sensitive botanicals such as calendula or arnica, with infusion settings as gentle as 110° F. for 6 hours. The system adjusts both thermal and agitation settings to reduce degradation of volatile compounds or delicate floral actives. These settings allow formulation of high-quality topical products with minimized oxidative loss or overprocessing.

In a further embodiment, the apparatus supports controlled melt protocols for various waxes and butters. Based on the “Melt Settings” table, the system can differentiate between materials like soy wax, beeswax, and carnauba wax, as well as dairy and palm-based butters. For instance, soy wax may be melted at 130° F. for 1.5 hours, while beeswax requires 150° F. for 1 hour. These melt parameters are executed through a dedicated module that prevents localized overheating and enables full liquefaction of the carrier prior to infusion or blending.

In an additional embodiment, the system includes a dedicated activation module for cannabinoid compounds, particularly useful for cannabis-based extractions. The “Activate Settings” demonstrate standardized protocols for activating THC and CBD from raw plant material, kief, or concentrates. For instance, dry flower activation for THC may be carried out at 240° F. for 45 minutes, whereas CBD-rich flower may require 60 minutes at the same temperature. These parameters are pre-programmed into the system and may be adjusted based on material form or user preferences.

In yet another embodiment, the system features selectable mixing profiles, both manual and automated. The “Mix Settings” data includes pulse modes for quick manual blending, 5-20 seconds depending on intensity, and auto-mix modes for continuous stirring. For example, the auto-mix function without heat runs 10-second agitation cycles every 30 minutes for up to 24 hours. This allows the system to maintain uniform suspension of materials in long-duration protocols without risk of thermal degradation.

In some embodiments, users may configure customized mix schedules, with fully user-defined durations, frequencies, and total runtimes. This customization is especially valuable for research or formulation of proprietary products, enabling granular control over blending processes. The control system logs all inputs and operational metrics, ensuring repeatability and traceability of each batch.

The system may also reference an internal efficiency database, as described in the “Herb Infusion Efficiency Table” sheet. This database maps known botanical materials, e.g., garlic, turmeric, cannabis, ginger, lavender, to estimated infusion efficiency values across different solvents such as oil, butter, glycerin, water, dairy, and honey. For example, curcumin in turmeric may have a 90% efficiency in oil or butter, but only 20% in honey. The infusion engine uses these values to calculate yield predictions and adjust potency estimates accordingly.

In related embodiments, the system draws on a bioavailability index to recommend the optimal infusion medium for each compound. The “Bioavailability index” sheet associates compounds like curcumin, CBD, allicin, gingerol, and linalool with their ideal and less efficient carriers. For instance, CBD is best delivered in oil or butter, while allicin from garlic infuses more efficiently in water. Suggested biological enhancers such as piperine, lecithin, or ethanol are also listed to improve absorption or extraction efficiency, providing valuable guidance for advanced users.

Together, these exemplary settings demonstrate the system's ability to execute finely tuned, compound-specific extraction and infusion routines. The modularity and flexibility across infusion, melt, activation, and mixing functions ensure compatibility with a wide range of organic materials and end-product applications. Whether preparing an edible, topical, tincture, or aromatic extract, the system provides a scalable, programmable platform to maximize potency, minimize waste, and improve product consistency.

In one embodiment, the infusion apparatus includes an indirect heating system comprising one or more external heating elements positioned in thermal communication with the processing chamber but not in direct physical contact with the chamber walls. The indirect heating configuration creates a controlled thermal environment that enables gradual and uniform heat distribution. This prevents localized overheating and protects temperature-sensitive compounds such as terpenes, cannabinoids, or volatile plant oils from thermal degradation. Unlike conventional decarboxylation systems that employ internal heating coils in direct contact with the material, the system disclosed herein achieves precision thermal regulation through spatial separation between the heating source and the infusion chamber.

The indirect heating elements are operably controlled via a set of external temperature sensors mounted adjacent to the chamber walls. These sensors are configured to monitor heat transfer in real time and relay temperature data to the system controller. The controller executes a feedback loop algorithm that adjusts heating intensity to maintain target thermal ranges within a margin of ±1.5° F. This level of control allows the system to execute long-duration infusion protocols without temperature spikes or fluctuations, thereby preserving the chemical integrity of the infused compounds and increasing overall process efficiency.

In some embodiments, the infusion system includes a potency estimation module operable to calculate the concentration of bioactive compounds within the final infused solvent. The potency estimation module may accept one or more user-provided input values such as material weight, declared compound percentage, infusion duration, heating temperature, and solvent volume. These inputs are used in conjunction with stored infusion efficiency factors specific to the selected botanical material and carrier medium. In one example, the system calculates both total compound yield and per-serving concentration, outputting results in milligrams per milliliter (mg/mL), milligrams per serving, or percentage by weight.

The module utilizes onboard algorithms and efficiency tables to estimate compound retention through each phase of the infusion process. For cannabis-derived materials, for instance, the system may apply a decarboxylation efficiency factor based on the heating profile used and further adjust for solvent-specific absorption rates. The module may dynamically revise potency estimates in real-time during processing by integrating actual chamber temperature readings and mixing durations. In some embodiments, the module is configured to display estimated potency trends graphically through the touchscreen or mobile application interface, allowing the user to visualize expected outcomes prior to final processing.

In one aspect, the infusion system includes a guided infusion module that prompts the user through each step of a selected infusion recipe. Upon launching a guided session, the user may be asked to input or confirm parameters such as material type, solvent, and desired potency. The system uses this input in conjunction with preloaded infusion profiles to automatically configure temperature, mixing intervals, and total duration. Throughout the cycle, the interface provides visual and/or audio prompts to assist the user in adding ingredients, confirming chamber status, and advancing through recipe stages.

The guided module further includes a dynamic feedback mechanism configured to monitor real-time sensor data during processing and compare current values against expected performance ranges. If the system detects a variance in temperature, time, or agitation cycle that may compromise the final potency, it generates an adaptive prompt instructing the user to extend the cycle, increase agitation, or otherwise correct the deviation. This live feedback enables the user to produce consistent, high-quality infusions without relying on external analytical tools, making the apparatus highly suitable for personal, clinical, or commercial use where dosage accuracy is critical.

In some embodiments, the infusion apparatus is in wireless communication with a mobile application operable on a computing device such as a smartphone or tablet. The application allows the user to remotely configure and monitor the apparatus in real time, providing full access to infusion profiles, operational controls, and process analytics. The interface includes configurable options for heating temperature, duration, agitation frequency, and cleaning cycles. Users may initiate, pause, or terminate infusion sessions from the mobile device and may receive push notifications when a process milestone is reached, or user input is required.

The mobile application is also integrated with the potency estimation module, allowing users to input desired outcome values and view calculated predictions before initiating the cycle. In some embodiments, the application is further configured to archive past sessions and support recipe creation with user-specific notes, dosage labels, and batch tracking. This mobile-based interaction increases accessibility and convenience while providing a secure and intelligent platform for managing infusion processes remotely. The mobile control architecture ensures that users can maintain consistency and safety regardless of their physical proximity to the device.

In one embodiment, the system includes an integrated auto-cleaning module configured to sanitize the processing chamber and internal fluid pathways following an infusion cycle. The module may include one or more internal spray nozzles operable to dispense heated water or cleaning solution into the chamber under controlled pressure. The cleaning cycle may be initiated manually by the user or automatically triggered upon completion of the infusion process, depending on user preferences or system settings.

The cleaning process may consist of multiple rinse cycles, including hot water flushing, agitation-assisted dispersion of cleaning agents, and final draining or evaporation of residual moisture. In some embodiments, the cleaning module also includes a temperature sensor to ensure that sanitation thresholds, such as 160° F., are reached and sustained for the appropriate duration. The auto-cleaning system minimizes manual maintenance, reduces cross-contamination risks, and supports repeated, high-frequency usage scenarios typical in commercial kitchens or healthcare environments.

In further embodiments, the system achieves an unexpected improvement in compound retention and infusion uniformity due to its sensor-regulated, indirect heating system. Unlike conventional systems that rely on embedded thermal coils or uncontrolled stovetop methods, the disclosed system provides tight control over thermal gradients within the processing chamber. This results in superior preservation of heat-sensitive molecules such as monoterpenes and flavonoids, which degrade rapidly above 150° F. in traditional devices.

The external sensor array enables continuous adjustment of heating power in response to real-time conditions, minimizing overshoot and thermal lag. This precision not only improves the safety and reliability of the process but also results in higher extraction efficiency and better product quality. Empirical testing shows that the system can maintain potency levels within ±5% of target values when operated under recommended profiles, an outcome not anticipated by prior art systems.

In one example embodiment, the indirect heating system includes an induction heating coil that encircles the base and side walls of the apparatus housing without making physical contact with the processing chamber. Heat is transferred through radiant conduction or magnetic field induction, depending on the material composition of the chamber. A glass or ceramic thermal barrier may be positioned between the heating source and the infusion vessel, thereby creating a buffered heating environment that prevents thermal hotspots. This embodiment is particularly advantageous for infusion of compounds that are sensitive to rapid temperature changes, such as CBD, terpenes, or floral extracts.

In another embodiment, the external sensors are configured as a distributed array placed at three or more equidistant positions around the outer perimeter of the chamber. These sensors generate continuous temperature data, which is averaged and transmitted to the processor in real time. This redundancy ensures fault tolerance and helps maintain uniform temperature throughout the chamber regardless of ambient fluctuations. In some variations, the system may allow users to toggle between standard and precision heating modes, where the latter activates tighter thermal regulation for delicate formulations.

In one example use case, the user inputs 2.5 grams of cannabis flower with an estimated potency of 18% THC into the system interface. The user selects olive oil as the carrier and enters a total infusion volume of 100 mL. Based on stored infusion efficiency values and decarboxylation loss factors, the system estimates a total available THC content of approximately 337.5 mg (after applying a 75% efficiency factor). The system then divides this total by the 100 mL volume to report a per-serving potency of 3.38 mg/mL. These values are displayed graphically on the touchscreen, along with an option to adjust serving size or dilution volume.

In another embodiment, the potency estimation module includes selectable input fields for different material formats such as dry flower, kief, concentrate, or pre-activated distillate. Each format has a unique infusion and activation profile stored in the system database. The system automatically modifies internal calculations to account for material format, expected volatility, and solvent-specific retention. This modularity allows users to confidently estimate output potency regardless of starting material type or preparation technique.

In one example of the guided infusion module, the user selects a preconfigured “Sleep Aid Tincture” recipe using chamomile, lemon balm, and MCT oil. The system guides the user step-by-step through material loading, solvent addition, chamber sealing, and setting confirmation. During the infusion cycle, the processor detects that the temperature is rising too slowly due to unusually cold ambient conditions. The system then displays a recommendation to extend the heating phase by an additional 15 minutes to ensure target potency is reached. The user confirms the prompt, and the process continues seamlessly.

In an alternative embodiment, the guided module is paired with a learning algorithm that adapts infusion settings based on user feedback. After completing a series of infusions, the system may ask the user to rate the strength or effectiveness of the result. These responses are logged and used to refine future protocol parameters for that user. Over time, the guided module evolves into a personalized infusion assistant capable of recommending not only parameter adjustments but also material and solvent pairings for specific use cases such as anti-inflammatory balms or sleep tinctures.

In one implementation, the mobile application provides a full-featured interface that mirrors the touchscreen display found on the physical apparatus. Through this interface, a user can initiate a new infusion session, review prior recipe logs, and receive alerts or estimated completion times. For example, a user at a dispensary may begin a decarboxylation process, leave the lab area, and monitor the status remotely through their smartphone. If a deviation is detected, such as lid misalignment or temperature delay, the app notifies the user instantly and provides correction options.

In another embodiment, the mobile application connects to a secure cloud database where users can upload and download community-created infusion recipes. Each recipe includes details such as compound concentration, herb-to-solvent ratio, infusion medium, and optional boosters (e.g., lecithin, piperine). The application may include filtering options by effect (e.g., energizing, relaxing), medium (e.g., oil, honey), or use-case (e.g., edible, topical). This networked ecosystem enables data sharing and protocol optimization across diverse user bases and supports use in both home and regulated environments.

In one embodiment, the auto-cleaning system includes a separate reservoir containing a food-safe cleaning solution that is automatically dispensed into the processing chamber upon activation. The system initiates a heat-assisted rinse cycle, using agitation to dislodge particulates and residue from the chamber walls and blades. A temperature sensor ensures that rinse water reaches a sanitation threshold of at least 160° F. before beginning the next phase. The spent fluid is drained into a disposable catch tray or routed to a built-in waste tank, which can be emptied manually.

In a variation of this embodiment, the user may schedule automatic cleaning cycles to occur after every infusion, once daily, or only after infusing specific materials known to be resinous or sticky. The cleaning parameters such as temperature, cycle count, and rinse duration may also be manually adjusted through the touchscreen or mobile application. This configuration is especially useful in commercial or clinical settings where regulatory compliance requires verifiable cleaning logs and consistent sanitation between product batches.

In one embodiment, the thermal sensor array is paired with a processor-executed predictive algorithm that anticipates temperature plateaus and adjusts energy input proactively. For example, during high-altitude use, where boiling points are reduced, the system may reduce power to prevent overshoot. Similarly, during cold-weather operation, the system may initiate a gradual ramp-up phase with stepped preheating stages to protect volatile compounds. These adjustments are invisible to the user but result in higher yield, improved product consistency, and reduced waste.

In another alternative embodiment, the system includes a “Thermal Trace Mode” that generates a graphical display of temperature over time. This graph may be used for quality assurance, protocol development, or academic research. Users can export temperature profiles via USB or wireless connection for inclusion in lab notes, compliance documentation, or standard operating procedures. This feature supports scientific validation of infusion results and provides an additional layer of transparency not found in consumer-grade devices.

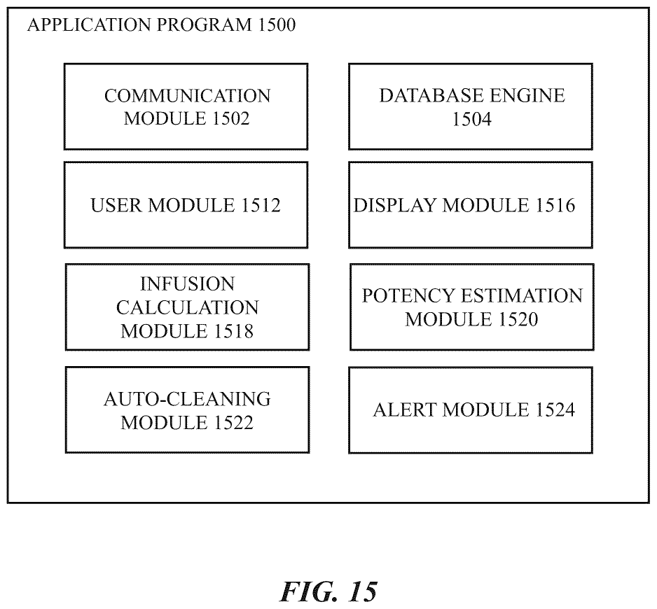

Referring now to FIG. 15, a block diagram is provided illustrating an exemplary architecture of the application program 1500 used in connection with the smart infusion apparatus described herein. The application program 1500 includes a communication module 1502, a database engine 1504, a user module 1512, a display module 1516, an infusion calculation module 1518, a potency estimation module 1520, an auto-cleaning module 1522, and an alert module 1524. These modules may be executed in part by a processor integrated within the apparatus, a companion computing device such as a smartphone or tablet, or a distributed system utilizing cloud-based components.

As further illustrated in FIG. 15, the potency estimation module is implemented as a discrete software component within the overall application architecture. The module communicates with the user interface through the display module, receives inputs via the user module, and queries the database engine for relevant potency and efficiency metrics. In one embodiment, the potency estimation module may be configured to update its logic and data tables via cloud synchronization, enabling the system to incorporate new compound profiles, updated extraction research, or regulatory standards. These updates may be triggered automatically upon system startup, or manually via the mobile app interface, and may include revised bioavailability factors, solubility curves, or suggested dosage ranges.

In some embodiments, the potency estimation engine is enhanced with machine learning algorithms trained on historical user data and system telemetry. After each completed infusion session, users may rate the perceived effectiveness, flavor strength, or texture of the final product. These ratings, together with known inputs such as temperature profiles, compound type, and session duration, are stored and analyzed to refine future potency predictions. Over time, this self-optimizing behavior allows the system to adjust its internal estimation models to reflect real-world outcomes, particularly in use cases involving novel ingredients or non-standard preparation techniques. This personalization layer ensures that potency guidance remains accurate and relevant to the individual user's preferences and formulation history.

Additionally, the system supports exporting potency estimation data as part of a final batch report. This functionality is particularly useful in regulated environments, small-batch manufacturing, or clinical applications where dosage consistency must be documented. Users may generate a printable or savable report that includes batch ID, preparation date, compound inputs, carrier details, calculated potency, and serving recommendations. These reports may be stored locally within the device memory, exported via USB or cloud storage, or shared directly from the companion mobile application.

The communication module 1502 is configured to establish and manage wired or wireless communication between the infusion apparatus and one or more external computing devices. In one embodiment, the communication module supports Bluetooth®, Wi-Fi®, and cellular protocols to enable connectivity with a mobile application. The module facilitates secure data exchange, including infusion profile settings, system status, alert messages, and user account information. In some embodiments, the communication module is also operable to connect to a remote server or cloud platform, permitting users to retrieve updated infusion protocols, firmware updates, or community-shared recipes.

The communication module 1502 also handles pairing and authentication between the apparatus and authorized users or devices. It may implement encryption protocols and multi-layer handshakes to ensure that sensitive data such as user preferences, potency calculations, and operational history are protected. In networked environments, the module may also transmit batch data and infusion logs to centralized databases for compliance, auditing, or research purposes.

The database engine 1504 is operable to store, retrieve, and manage data necessary for the functioning of the application program 1500. In one embodiment, the database stores infusion profiles, herb-specific potency metrics, infusion efficiency tables, and bioavailability indexes. The database engine is optimized for low-latency read and write operations and may support both structured and unstructured data models. The system may include local caching of high-priority records to reduce network dependency, and periodic synchronization with cloud-based repositories.

In another embodiment, the database engine 1504 allows user-defined data entries such as custom profiles, notes, and historical performance logs to be stored and retrieved. Data stored in the database may be cross-referenced by tag, date, herb type, or use-case category. This relational structure allows for robust querying and analytics, supporting advanced features like session comparison, batch tracking, and performance scoring. The database engine interfaces directly with all other modules to ensure that relevant data is available in real-time.

The user module 1512 is responsible for managing all user interactions within the application program. This module enables profile creation, authentication, preference management, and history tracking. In one embodiment, the user module includes tiered permission settings, allowing for different access levels such as administrator, standard user, or guest. These settings may control which users can modify infusion parameters, initiate cleaning cycles, or access stored potency logs.

The user module 1512 may also handle scheduling features, allowing a user to preset an infusion to begin at a future time. Notifications and saved preferences are linked to individual user profiles, enabling continuity across devices and across multiple uses. In shared or commercial environments, this module ensures accountability and individualization of results by associating each infusion session with a specific user ID or device credential.

The display module 1516 governs the graphical user interface (GUI) of the application program, rendering visual elements such as menus, input forms, real-time process updates, and graphical data representations. In one embodiment, the display module supports swipe navigation between screens as illustrated in FIG. 14, allowing the user to transition seamlessly between different operational functions such as “Infuse,” “Melt,” “Mix,” “Activate,” and “Calculate.” The module also supports dynamic content rendering, wherein process metrics such as temperature, mix duration, or potency estimates are displayed in real-time.

The display module 1516 is integrated with accessibility and customization settings. For example, users may toggle between light and dark modes, switch units between Fahrenheit and Celsius, or adjust font sizes for improved readability. The display module interfaces with the user module to reflect user-specific preferences and may also respond to alert module triggers by displaying notifications, warnings, or prompts directly within the interface.

The infusion calculation module 1518 is configured to calculate expected process durations, energy consumption, and compound yield based on user-selected parameters and stored efficiency values. In one embodiment, the module uses predefined equations and lookup tables to predict total infusion time, solvent saturation point, and mixing requirements. The calculation module takes into account the selected material form (e.g., dry flower, kief, or extract), solvent type, batch volume, and desired output concentration.

In some embodiments, the infusion calculation module is responsive to real-time data from the apparatus, such as temperature slope, mixing blade load, or elapsed time, allowing it to refine its calculations dynamically. If the system detects that heating is slower than expected, the module may recalculate the remaining time or suggest extending the infusion duration. These dynamic adjustments provide a layer of intelligence that compensates for environmental or material variability, improving final product consistency.

The potency estimation module 1520 is operable to provide the user with a calculated estimate of both total and per-serving potency based on user input and sensor data. In one embodiment, the module receives variables including material weight, compound concentration, infusion time, solvent volume, and target serving size. The module then applies known or empirically derived extraction efficiency and decarboxylation factors to generate a compound yield prediction. Results may be displayed as total milligrams of active compound per batch and per defined dose.

In alternative embodiments, the potency estimation module may incorporate real-time thermal profiles from the device and adjust estimates based on actual temperature readings and exposure times. This feedback-driven model ensures greater accuracy compared to static calculators. The module may also allow for exporting potency reports for labeling, recordkeeping, or quality assurance. In medical or therapeutic settings, this feature supports dosage control and regulatory compliance.

The auto-cleaning module 1522 manages the automated sanitation sequence of the device following completion of an infusion cycle. In one embodiment, the module triggers a rinse cycle using heated water or a food-safe cleaning solution dispensed via internal nozzles. The cycle may include a soak stage, agitation, and drain sequence, all controlled by pre-programmed timing and sensor feedback. The cleaning module may operate as a standalone routine or be scheduled to follow specific operations, such as infusions involving resinous materials or allergens.

In another aspect, the auto-cleaning module monitors key indicators such as temperature during cleaning, flow rate of rinse fluids, and completion of each phase. If an anomaly is detected, such as a blockage or insufficient fluid level, the module may halt the cycle and notify the user via the alert module. Additionally, cleaning cycles and durations may be logged into the database engine for reference, particularly in shared-use or regulated environments requiring sanitation documentation.

The alert module 1524 is configured to issue notifications to the user during all phases of device operation. Alerts may be visual, auditory, or pushed to a connected mobile device via the communication module. In one embodiment, alerts include process completions, ingredient addition prompts, safety warnings (e.g., high temperature), and cleaning cycle confirmations. The alert module integrates directly with the display and user modules to deliver context-specific instructions or status reports.

In more advanced implementations, the alert module may also serve a diagnostic function by issuing error codes or fault messages in response to system irregularities. For example, if a sensor returns out-of-range values or if the lid is detected as open during heating, the alert module immediately notifies the user and pauses operation. Alerts may be stored in the database engine and associated with the session ID for subsequent review, contributing to transparency and traceability of device use.

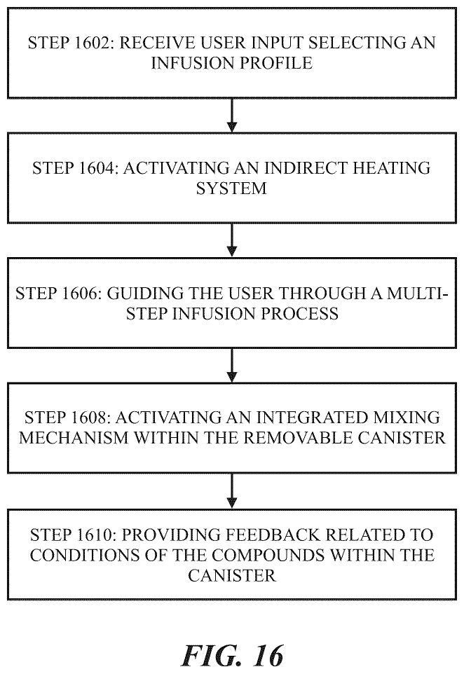

Referring now to FIG. 16, a flowchart is provided illustrating an exemplary method for performing a multi-step guided infusion process. The method is implemented using the smart infusion apparatus in combination with the application program, and it leverages integrated subsystems including the heating system, mixing mechanism, user interface, and feedback modules.

The method begins in step 1602 by presenting the user with an interactive interface, either on the apparatus touchscreen or on a connected mobile application, where the user may select from a list of infusion profiles. Each profile corresponds to a specific botanical material and solvent pairing, and is configured with recommended temperature, duration, and agitation settings. The user may select a preset profile (e.g., “Topical CBD in MCT Oil”) or manually configure a custom profile. The interface may further allow input of target potency, material weight, solvent volume, and serving size to inform subsequent calculations.

In step 1604 and upon confirmation of the selected infusion profile, the system activates the indirect heating module, which applies thermal energy to the infusion chamber without direct contact between the heating elements and the canister walls. External temperature sensors positioned around the canister continuously monitor temperature conditions and transmit data to the processor. A feedback control loop adjusts power delivery in real time to maintain the specified thermal range throughout the cycle. This ensures uniform heat application, minimizes overshoot, and preserves the integrity of thermally sensitive bioactive compounds.

In step 1606 and as the infusion process proceeds, the system guides the user through multiple discrete steps. For example, the user may first be prompted to load the organic material, followed by the addition of a specific volume of solvent after preheating. Prompts are displayed on-screen and may be accompanied by audible alerts or vibration notifications via a mobile device. In some embodiments, the system verifies that required actions have been completed through sensor inputs, such as lid closure confirmation, weight detection, or temperature validation, before advancing to the next step.

In step 1608, after all ingredients have been added, the system initiates the mixing cycle using an integrated blade or agitation mechanism housed within the canister. The mixing cycle may be continuous, pulsed, or periodic, depending on the selected infusion profile. For example, a profile for delicate herbs may use 2-second pulses every 30 minutes, while a robust root extract may require high-frequency blending. The mixing mechanism helps dissolve and disperse the active compounds evenly throughout the solvent, resulting in consistent compound distribution across the entire batch.

In step 1610, the system continuously monitors and displays real-time operational metrics including current chamber temperature, time remaining in the cycle, and estimated potency of the infusion. The potency estimation is dynamically updated using inputs such as process duration, temperature exposure, solvent type, and material weight. These values are presented on the interface in both graphical and numerical formats, enabling the user to assess infusion progress and make informed decisions about adjustments or extensions. Upon completion, the system may generate a report or log summarizing the infusion session for recordkeeping or compliance purposes.

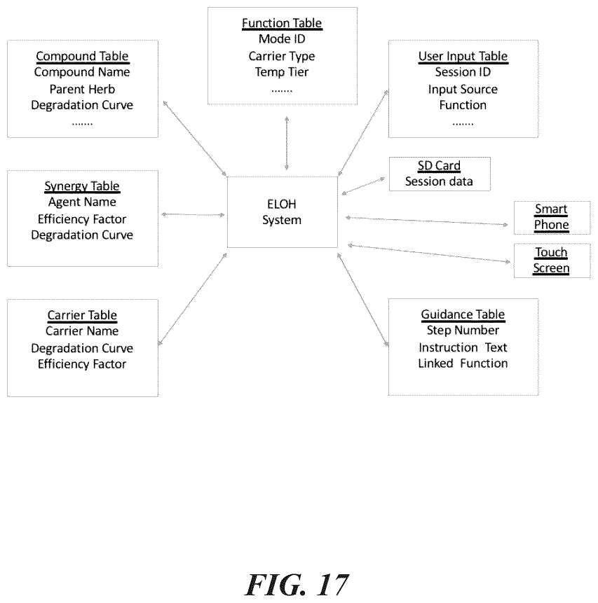

Referring now to FIG. 17, a block diagram is shown illustrating an exemplary database schema architecture for the ELOH system. The system architecture is structured to facilitate both real-time and historical data retrieval, enabling the smart infusion device to operate in a data-informed and context-aware manner. At the core is the ELOH system database, which interacts with a plurality of relational tables including a compound table, synergy table, carrier table, function table, user input table, and guidance table. These tables are connected bidirectionally to the central database engine, ensuring all functional modules of the system have access to up-to-date compound-specific, user-specific, and session-specific information.

The Compound Table includes entries for individual compounds such as CBD, curcumin, or allicin. Each entry may contain associated data fields including the parent herb source, degradation curves across temperature or time intervals, and solubility or carrier compatibility. The Carrier Table and Synergy Table store data related to extraction efficiency factors, degradation profiles, and potential interaction effects of co-administered agents or carrier liquids. For instance, turmeric's curcumin may have an efficiency factor of 0.9 in oil and 0.2 in water, with degradation initiated at temperatures exceeding 145° F. These empirical values are accessible to the calculation and estimation engines during process planning.

The Function Table defines infusion parameters linked to predefined or user-created modes, including mode ID, carrier type, and temperature tier (e.g., low, medium, high). The User Input Table logs session data including source of user input (e.g., touchscreen or smartphone), function selection, and timestamps. Data may also be stored or backed up to an SD card for portability or batch record retention. The Guidance Table houses the step-by-step instruction sequences used in the interactive infusion guides, mapping step numbers to instruction text and linking each to a corresponding function within the system. This architecture ensures modularity and expandability, allowing new materials or infusion protocols to be integrated without altering the core platform logic.

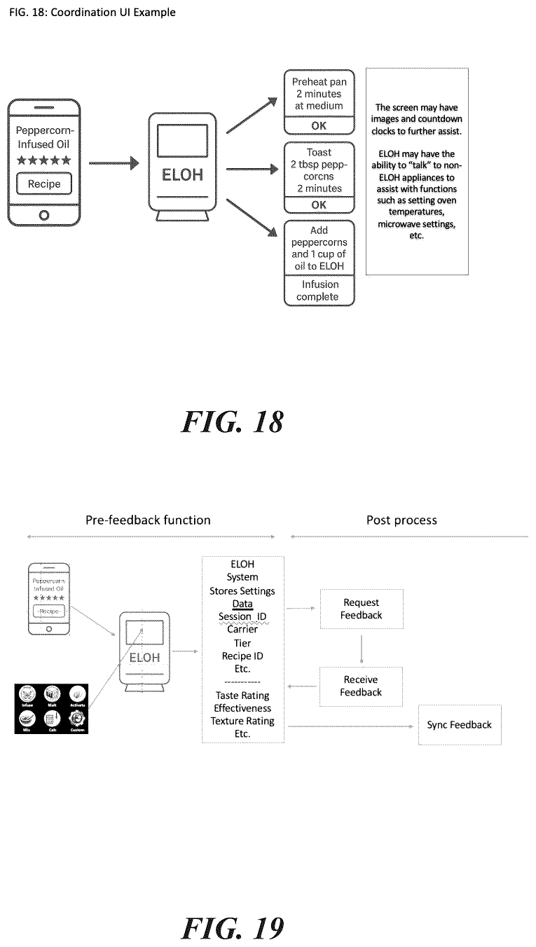

FIG. 18 illustrates an exemplary coordination user interface used to synchronize procedural steps between a user's mobile device and the ELOH infusion appliance. In one embodiment, the user selects a recipe or guidance protocol from their mobile application, such as “Peppercorn-Infused Oil,” and initiates the process. The ELOH system then coordinates the timing and sequencing of steps that may extend beyond the device's internal heating or mixing capabilities. For example, the user may first be prompted to preheat a pan for 2 minutes at medium heat, then toast peppercorns for a specified duration, before transferring ingredients into the ELOH unit for infusion.

The coordination interface is designed to bridge multiple kitchen appliances and manual steps into a seamless guided workflow. The interface may include countdown clocks, image overlays, and confirmation prompts to guide the user through each step with clarity and precision. Furthermore, the ELOH system may be configured to “communicate” with other smart appliances over local protocols or cloud APIs. For example, the system may automatically preheat a smart oven, initiate a microwave timer, or sync with a kitchen scale to measure ingredients. This functionality transforms the infusion process from a single-device task into a full kitchen orchestration experience, extending the utility of the system into broader culinary and functional wellness applications.

FIG. 19 illustrates an exemplary user feedback loop that enables performance refinement of infusion processes through real-time and post-process user input. The loop begins with the user selecting or pushing a recipe to the ELOH device from their smartphone. Upon initiating the infusion process, the system records session-specific data including the selected carrier, infusion tier, time-temperature profile, and a unique session ID. This data is stored within the ELOH database and associated with a specific recipe ID or user profile.

After the process concludes, the ELOH system prompts the user for feedback through either the device interface or a mobile application. Feedback fields may include qualitative metrics such as taste rating, texture consistency, effectiveness of infusion, or user satisfaction. Once received, the feedback is synced back into the ELOH database, where it is indexed by session ID and used to inform future firmware updates, calibration algorithms, and recommended presets. For example, if a recipe consistently receives lower potency feedback under a certain profile, the system may recommend a higher temperature tier or longer duration for future sessions. This continuous feedback loop allows the device to self-optimize over time and personalize operation to suit specific user needs or compound characteristics.

The system includes a removable canister constructed from food-safe, heat-resistant materials. Canisters may vary in volume, geometry, or thermal mass to support different batch sizes, heating profiles, or mixing intensities. Additional features may include anti-rotation structures, visible volume markings, interchangeable lids, sealed plugs, or vented configurations to support different use cases. The canister is designed for safe handling and compatibility with a wide range of ingredients, including botanical, culinary, cosmetic, nutraceutical, or functional materials, and is reinforced to tolerate both low and elevated temperature conditions.

Heating is delivered through indirect radiant or conductive means. Depending on the canister design and intended application, the system may use resistive, convective, or inductive heating elements. One or more external sensors monitor ambient or adjacent conditions to support closed-loop heat modulation. The system is optimized for low-temperature compound preparation typically operating below 300° F., and is suitable for workflows involving thermally sensitive compounds, such as cannabinoid decarboxylation, curcumin retention, or preparation of other bioactive ingredients vulnerable to heat degradation.

The canister supports an integrated or coupled mixing mechanism. This mechanism may include interchangeable blades or stirrers suited for blending, emulsifying, or homogenizing different material types. Mixing can occur independently or alongside heating, enabling operation modes such as room-temperature infusion, maceration, or shear-driven dispersion. In some embodiments, mixing cycles may be customized for continuous or pulsed motion to support formulation goals such as uniform particle suspension or compound distribution.

The processor executes adaptive control algorithms using system inputs like user settings, sensor feedback, and ingredient parameters. Inputs may include calculated weight from a potency estimator or an integrated scale. Additional data may derive from scanned identifiers, lab results, or references like USDA nutrient databases. These inform thermal and mechanical control, enhancing formulation consistency and potency via a logic database (e.g., ELOH framework), which enables adaptive workflow execution.

In some embodiments, the system includes a user interface designed to facilitate guided interaction and workflow customization. The interface may be a touchscreen, external display, or companion mobile application, and it allows users to select process goals (e.g., “Infuse,” “Melt,” “Activate,” or “Mix”) from a menu of predefined or customizable options. Once a function is selected, the processor may prompt the user for material type, target batch size, or intended outcome (e.g., Gentle, Balanced, or Bold infusion), and adjust process parameters accordingly. These adjustments may reference the logic database, which contains stored process models and best-practice parameters. Where the logic database is implemented as ELOH, the system may deliver real-time prompts, including timing suggestions, safety warnings, or quality checks, based on internal status and user selections. The interface may support both simplified presets and expert-level configuration, reducing cognitive burden while improving safety and consistency across a range of user skill levels.

In some embodiments, the ELOH may assist in managing steps that occur outside of the unit. For example, in making coriander garlic oil, the coriander seeds may be toasted first. The ELOH may then advise users (via the display, timers, confirmation requests, etc.) through the process. In such the ELOH, in a way, navigates the user through the various processes performed by the invention.

The system supports wireless communication via Wi-Fi and Bluetooth, with optional NFC or other proximity-based protocols in future versions. These interfaces enable device pairing, firmware updates, and cloud-based recipe synchronization through a companion mobile application. A physical data port may also be included for accessories or offline firmware or recipe updates. In some embodiments, the system may coordinate timing, prompts, or workflows with external appliances, enabling guided execution of cross-device routines that support compound integrity and user convenience.

The system supports dual-voltage operation (e.g., 110V and 220V) for international deployment. In some embodiments, voltage compatibility is achieved using an auto-sensing power board. Alternatively, a switchable transformer, external regulator, or adaptable interface may be used to meet local requirements. Firmware configurations may localize operational presets, safety limits, and language preferences. Physical plug adapters or region-specific cords may be included or offered separately to support global distribution. This combination of hardware flexibility and software adaptability enables consistent system performance across regional power and language standards. In some embodiments, the processor may also adjust heating profiles or cooldown timing based on detected input voltage, maintaining consistency of results regardless of regional conditions.

As used herein, “software-defined preparation modes” refers to workflows programmed via ELOH algorithms, customizable through the touchscreen interface to execute specific preparation sequences (e.g., infusion, melting, activation, mixing) based on user inputs and sensor feedback to deliver compound-specific processing profiles.

As used herein, the term “modular processing” refers to the system's ability to execute preparation workflows using interchangeable physical components, such as canisters, blades, or inserts, together with customizable, software-defined steps that may be sequenced according to user goals. For instance, a user may replace a 1-liter stainless steel canister with a perforated 0.5-liter steel canister equipped with a vented lid for enhanced airflow and moisture escape, then select a drying or activation profile optimized for water reduction at low to moderate heat (e.g., 180° F. for 1.5 hours). Alternatively, a sealed glass canister with a vapor-capture adapter may be selected to condense and retain aromatic volatiles during infusion. This flexibility enables adaptation to different ingredients, volumes, and processing objectives without requiring changes to the base unit's firmware or structural configuration, while maintaining precise control over timing, temperature, and agitation.

In its current embodiment, the system is optimized to produce compound-rich infusions that can be integrated into larger meal or formulation routines. This reflects a strategic use case: enabling users to incorporate health-supportive compounds (e.g., curcumin, gingerol, cannabinoids, essential oils) into everyday cooking or therapeutic workflows with greater consistency, safety, and dosing confidence. Some embodiments may include optional vapor-capture vessels designed to condense and retain volatiles during processing, supporting applications in perfumery, distillation, or scent preservation.

The term “coordination” refers to the system's ability to guide user actions or synchronize steps that may involve external appliances or manual tasks, regardless of whether those devices are directly controlled. Coordination may be achieved via wireless communication (e.g., Wi-Fi, Bluetooth, or NFC) when supported, or through sequenced prompts instructing the user to perform external steps, such as preheating an oven, pan-searing an ingredient, or staging containers. Example workflows include: (a) activating a botanical ingredient in the device, then prompting the user to add it to a soup or sauce prepared elsewhere; (b) creating an infused oil intended for use with roasted vegetables or proteins cooked separately; or (c) guiding a multi-phase recipe where an infused base is created in the system and then combined with ingredients processed through other appliances. These workflows may be supported by the ELOH framework or executed independently, enabling the system to serve as a preparation engine within multi-appliance, health-forward routines.

The potency estimation logic relies on the ELOH database (Empirical Logic Organizing Heuristics), which stores values for compound efficiency, carrier compatibility, degradation thresholds, and solubility. This database uses a lightweight relational schema (e.g., SQLite) and contains structured entries for carrier-compound pairings. Each entry may include fields such as compound name, form (e.g., dried, fresh, extracted), estimated efficiency under standard processing conditions, and known synergies (e.g., piperine with curcumin). Data may originate from published literature, in-house testing, or third-party validated sources, and is updated via firmware or app-based cloud sync to ensure that potency guidance reflects the most current available standards. As used herein, ‘transformative agent’ refers to compounds like piperine, terpenes, or citric acid that enhance extraction, activate precursors, or improve bioavailability, as stored in the ELOH database.

The system uses the ELOH database to estimate compound potency and bioavailability based on ingredients, carriers, settings, and transformative agents. For example, when dried turmeric is selected, the system calculates curcumin content (e.g., 2-5% by weight, USDA data), applies a 30% loss factor for low-temperature infusion (Anand et al., 2007, Molecular Pharmaceutics), and uses a 50-70% bioavailability factor for lipid carriers (e.g., MCT oil) to estimate yield (e.g., 126 mg per 100 mL for 10 g turmeric). If the user confirms adding piperine (0.05-0.2 g), bioavailability rises to 80% (Shoba et al., 1998, Planta Medica), yielding 168 mg per 100 mL or 8.4 mg per 5 mL serving. For mushroom-based formulations, such as elixirs or teas, citric acid boosts polysaccharide extraction by 10-20% (Wang et al., 2017, Food Chemistry), with user confirmation prompting adjusted potency estimates.

For aromatherapeutic candles, the system uses the ELOH database to suggest fragrance oil concentrations (e.g., 6-10% w/w) and stabilizers like terpenes (e.g., limonene, 0.5-1 g per 100 g wax) based on user-selected fragrance strength (e.g., mild, moderate, strong) for a wax candle (e.g., soy, paraffin, or other waxes) at a suitable temperature based on wax type. The system estimates scent potency or strength, factoring in limonene's 5-10% increased retention (Adams et al., 2010, Journal of Agricultural and Food Chemistry), with user confirmation via prompts adjusting estimates for enhanced scent retention.

In embodiments where the logic database is implemented as ELOH, fixed numerical values may be stored for each compound-carrier pair, such as curcumin content (3%), degradation loss (30%), and MCT solubility (60%). These values may be revised periodically via cloud-sync based on validated references, including USP monographs, peer-reviewed studies, or manufacturer specifications. The system may use these values to recommend optimal carriers based on solubility profiles, enhancing extraction efficiency and overall compound yield.

In some embodiments, the system supports barcode scanning, QR code scanning, and/or Arrhenius kinetics for automated ingredient recognition, workflow configuration, or potency estimation. Scanning may be performed via a companion mobile device or, in certain versions, directly through an integrated camera located behind the touchscreen interface. When connected to Wi-Fi or a local network, the system may retrieve compound, recipe, or processing data by decoding GS1-standard barcodes (e.g., UPC, DataMatrix) and querying a structured endpoint via a RESTful or GraphQL API. Example endpoints include manufacturer-maintained ingredient libraries, verified compound registries, or centralized knowledge services operated by the manufacturer or an authorized third party.

Data packets may be formatted in JSON and include fields such as compound name, concentration, ingredient form, geographic origin, or workflow parameters. Retrieved data is parsed by the processor to auto-fill input fields, match stored workflow presets, configure sequences involving multiple ingredients (e.g., botanical, fungal, or carrier), and activate real-time guidance for safety or timing. These functions may be accessed through the touchscreen interface or companion mobile application.

The system may present alerts and task prompts through the touchscreen interface, audible notifications, or synchronized messages on a connected mobile device. These may include internal system status updates, such as “target infusion temperature reached” or “cooldown complete”, as well as instructions for manual or external actions, such as “preheat oven to 350° F.” or “begin stovetop step.” Where supported, these prompts may be mirrored to connected smart appliances using wireless protocols. Even in the absence of direct appliance integration, the system can deliver sequenced instructions based on internal timing logic. These instructions may be accompanied by visual indicators, such as progress bars, countdown timers, or step-by-step icons, to facilitate user adherence to multi-stage workflows and reduce operational ambiguity.

To enhance operational safety, the system may include interlock mechanisms that prevent blade rotation unless the lid is properly secured. In some embodiments, heating may pause or stop if the lid is removed mid-cycle. Additional safeguards may monitor for abnormal temperature spikes, sensor failure, or power disruption, triggering shutdowns or initiating cooldowns to protect both compounds and users. Integrated cooling fans may engage automatically after high-temperature tasks to reduce surface heat and preserve sensitive materials. These protections may be tied to user profiles or preparation modes, enabling customization while maintaining baseline safety across all workflows.

The system incorporates safety features that protect both the user and the integrity of heat-sensitive compounds. These include auto-cooldown cycles, maximum temperature limits (e.g., 300° F./149° C.), and a child-lock function to restrict interaction during and after processing. The canister may include an insulated handle designed to remain cool during operation and facilitate safe pouring of hot materials, such as molten wax or infused oils. In some embodiments, a heat-shielded sleeve or cradle may be included to assist with handling during high-temperature transfers. The canister's shaped spout provides directional flow to reduce splatter or spill risk. Following thermal operations, cooling fans may engage automatically, and the interface may alert users when it is safe to handle or pour contents. Certain operations may be restricted unless safety confirmations, such as cooldown timers, lid engagement checks, or manual acknowledgments, are completed.

To improve user comfort and discretion, the system may include passive or active odor control. A sealed plug, made from heat-resistant material such as silicone, limits vapor escape during heating. In some embodiments, modular accessories may support enhanced odor management via replaceable and/or removable filters containing activated carbon or other neutralizing media. The lid and plug design may direct vapors along a defined path, enabling optional attachments for deodorizing, condensing, or diffusing purposes. In some embodiments, the system may utilize negative-pressure odor management systems using the fans and vents in the system. These features enable low-odor operation even when processing aromatic or high-volatility compounds, enhancing discretion and environmental comfort during home or small-batch use.

The system supports firmware-based updates that enable new preparation modes, ingredient presets, interface enhancements, or workflow guidance features. Updates may be delivered via Wi-Fi, Bluetooth, NFC, or physical media. The update process is designed to be secure and user-friendly, with on-screen prompts and optional mobile notifications. In some embodiments, firmware updates may incorporate previously saved user preferences, session profiles, or device usage patterns to deliver personalized features.

The system may include a real-time clock and session logging functionality to track preparation history, including ingredient type and quantity, temperature, and duration. Timestamps and batch data may support scheduling, consistency, and integration with connected appliances. Users may name and save session profiles for repeat use. In app-connected systems, these sessions may sync across devices and generate trend summaries or alerts. Over time, the system may analyze session feedback, such as user ratings, preparation outcomes, or perceived potency, to recommend process adjustments, refine presets, or train personalization models that improve outcome predictability.

The canister is designed for repeated use and easy cleaning. In some embodiments, the system supports a rinse or self-cleaning mode. Canisters may be made from dishwasher-safe materials or smooth-surfaced composites to minimize residue buildup. Interchangeable canisters may vary in volume or surface treatment to support different ingredients or batch sizes. The system may identify canister type through mechanical indexing, sensors, or user input, enabling automatic adjustment of settings.

In some embodiments, the system coordinates external appliances or manual tasks using protocols like MQTT or RESTful APIs to transmit JSON-formatted instructions via system interfaces, such as a mobile application or touchscreen. For example, it may command a smart oven to preheat to 350° F., guide stovetop toasting of peppercorns prior to infusion, or guide baking for external recipes. Where appliances lack connectivity, visual or audible prompts align with internal timing. These features support multi-appliance workflows, complementing internal ELOH-driven processes and reinforcing compound integrity.

Where supported, smart appliances receive direct control signals integrated with processor-programmed workflows (e.g., those informed by a logic database such as ELOH). Where unsupported, instructions are displayed as visual or audible prompts aligned with internal timing or uploaded recipe steps. These coordination features extend the system's guidance role into multi-appliance, recipe-driven workflows, reinforcing compound integrity across culinary, cosmetic, aromatherapeutic, and wellness formulations.

The system may support a tiered functionality model in which core presets are available by default, while advanced features, such as guided recipes, batch analytics, or AI-enhanced workflows, may be accessed through subscription or one-time purchase. Users may opt to share anonymized usage data, including session logs and batch ratings reflecting characteristics such as taste, aroma, texture, potency, or perceived effectiveness. These ratings may be structured (e.g., 1-5 scale) or freeform (e.g., notes entered via the app), and can be aggregated to improve default presets and support algorithmic refinement. Over time, this feedback may inform personalization models, cross-device coordination, and the design of future product features or workflows.

The system supports diverse workflows for botanical, fungal, or carrier formulations, including ingestible blends (e.g., syrups) and non-ingestible products (e.g., aromatherapeutic candles, balms). Candles use fragrance oils stabilized with terpenes (e.g., limonene) at a suitable temperature based on wax type in modular canisters to enhance scent retention. Mushroom-based wellness formulations, such as elixirs or teas, employ citric acid for polysaccharide extraction at a suitable temperature (e.g., 140-200° F.). Modular thermal control and guided presets facilitate accurate control across applications.

The system may support interface-level modularity through swappable display panels, smartphone docks, or embedded mobile devices configured to run the control interface. In some embodiments, external accessories, such as specialized lids, mixing attachments, or heating bases, may be detected automatically by the processor, triggering context-specific presets or visual instructions. These hardware extensions enable the system to expand its capabilities over time without requiring wholesale replacement. This supports flexible adaptation across culinary, wellness, and other compound preparation workflows.

The system may support personalized user profiles stored locally or synchronized via a companion application. Profiles may include saved presets, ingredient preferences, usage history, and interface customizations. In some embodiments, profiles may also control access to restricted features, such as child-safe modes, high-temperature operations, or age-restricted compound workflows, and may be secured using a PIN code, biometric input, or linked device authentication.

Example compound preparations include turmeric oil with piperine, CBD-infused balms, ginger syrups, and aromatherapy candles. Each formulation may require different heating, mixing, or hold profiles. The system executes these variations using modular presets, guided steps, or user-defined sequences. For instance, a wax-based candle may involve high-temperature melting without agitation, while a ginger syrup may require extended low-heat infusion with intermittent stirring. The system's profile-based logic ensures accurate and repeatable outcomes across diverse use cases.

The system is designed for use with heat-stable, food-grade materials suitable for culinary, wellness, and topical applications. Components such as canisters, blades, and lids may be manufactured from anodized aluminum, stainless steel, heat-resistant polymers, or borosilicate glass. The removable canister is engineered to tolerate sustained temperatures above 300° F. (149° C.), with structural reinforcements such as double-walled construction, reinforced flanges, or thermally resistant seams. Operational protocols, however, limit in-use temperatures to a maximum of approximately 300° F., not due to hardware constraints, but to preserve compound integrity by avoiding uncontrolled or excessively high thermal exposure common in conventional heating systems. The system may detect incompatible components and alert the user. Optional canisters without blades may support workflows such as wax melting or passive heating. Visible fill lines or volume indicators may be included to prevent overfilling and ensure consistent performance.

To support international use, the system may include a dual-voltage power supply or auto-switching circuitry compatible with 110V and 220V. In some embodiments, the processor may adjust heating profiles or cooldown timing based on input voltage to maintain consistent results across regions. Firmware or UI language settings may also be localized to support regional deployment.

User data, including session logs, profile settings, and downloaded presets, may be stored locally or synchronized via encrypted transmission to a secure cloud environment. Users may opt in to anonymized data sharing to support ecosystem improvements. Personally, identifying information is not stored by default, and access to user data may be revoked or re-authenticated in the event of device loss, resale, or account migration.

The system integrates indirect heating, guided mixing, potency estimation, and modular hardware to simplify compound-rich formulations across culinary, wellness, and cosmetic domains, preserving compounds like curcumin or fragrance oils. Sequenced prompts and personalization ensure controlled dosing and consistent outcomes.

What is claimed is:

1. A smart system for preparing infused, melted, activated, or blended culinary, cosmetic, aromatherapeutic, and wellness botanical, fungal, and carrier formulations, comprising:
a removable canister configured to receive botanical, fungal, or carrier materials, thermally insulated from direct-contact heating;
an indirect heating system comprising external sensors and thermal control logic, configured to regulate internal temperatures at a maximum suitable for compound integrity based on sensor feedback;
a mixing mechanism mechanically coupled with the canister, operable at elevated temperatures, comprising interchangeable blade assemblies or blade-free configurations for agitating, blending, emulsifying, or homogenizing contents according to programmable mixing cycles;
a processor configured to execute database-programmed workflows, coordinating multi-stage thermal and mechanical preparation based on a logic database comprising compound efficiency, solubility, degradation, and synergistic interactions for transformative agents, such as stabilizers, extractants, or enhancers;
a touchscreen interface operable locally or via a mobile application, configured to enable user selection of preparation modes, including aromatherapeutic candles or mushroom formulations, and display guidance and potency or strength estimation, including scent strength, based on database parameters and input parameters, including sensor feedback;
a cooling mechanism with fans to accelerate cooldown and preserve heat-sensitive compounds;
one or more guided prompts restricting progression without user confirmation.

2. The system of claim 1, wherein the mixing mechanism supports stirring, emulsifying, high-shear homogenization, or passive melting, selectable for culinary recipes, cosmetic balms, aromatherapeutic candles, wellness tinctures, or botanical processing.

3. The system of claim 1, wherein the potency estimation module references a database with fields for compound efficiency, solubility, degradation coefficients, and synergies, including those with botanical compounds, fragrance oils, or fungal materials, updated via cloud sync, wherein the potency estimation module permits modular operations to enable a plurality of modes to be operated independently or in a predefined sequence.

4. The system of claim 1, wherein the cooling mechanism engages automatically above a threshold with interlock mechanisms restricting operation until safe conditions are detected.

5. The system of claim 1, wherein the canister is dishwasher-safe with volume markings and supports workflows including cosmetic balms, aromatherapy candles, standalone carrier melting, or mushroom-based wellness formulations, such as elixirs, teas, or extracts.

6. The system of claim 1, wherein the canister includes structural features and interchangeable lids with seals or vents, constructed from food-safe, heat-resistant materials.

7. The system of claim 1, wherein the formulations comprise bioactive compounds and carriers, including cannabinoids, curcumin, gingerol, polysaccharides, oils, waxes, glycerin, syrups, alcohols, or chocolates.

8. The system of claim 1, wherein the database-programmed workflows dynamically adjust parameters based on material properties, such as solubility, degradation, or volatility, for botanical activation, fungal extracts, aromatherapeutic oils, cosmetic balms, or wellness tinctures.

9. The system of claim 1, wherein the canister supports small to medium batches, typically up to 1.5 liters or greater, depending on configuration.

10. The system of claim 1, wherein the system supports dual-voltage operation (e.g., 110V and 220V) with auto-switching circuitry or localized firmware settings.

11. The system of claim 1, wherein user data, including session logs and presets, is stored locally or synchronized via encrypted cloud transmission.

12. The system of claim 1, wherein the database-programmed workflows enable user confirmation of a transformative agent, such as stabilizers, extractants, or enhancers, adjusting potency or strength estimates based on database parameters for extraction efficiency, activation, bioavailability, or scent retention.

13. A method for preparing a compound formulation, the method comprising the steps of:
receiving user input via the touchscreen or mobile application to select a database-programmed mode for botanical, fungal, or carrier formulations, including transformative agents like stabilizers or extractants;
initiating a heating cycle with a calculated temperature profile;
executing synchronized mixing operations;
estimating potency or strength, including scent strength for candles, using database parameters; and
initiating a fan-driven cooldown sequence.

14. The method of claim 13, wherein potency or strength estimation applies database parameters for infusion time, carrier type, and input potency, optimizing yield predictions with synergies, including those with botanical compounds, fragrance oils, or fungal materials.

15. The method of claim 13, wherein sensor data is sampled regularly to adjust parameters, preserving compound integrity.

16. The method of claim 13, wherein the process includes:
thermally activating compounds without carriers;
melting carriers;
infusing carriers with botanical or fungal materials; and
blending for homogeneous formulations.

17. The method of claim 13, further comprising receiving user input for compound type or goal, modifying parameters.

18. The method of claim 13, wherein the cooldown sequence uses real-time temperature data to restrict actions until safe thresholds, signaled by alerts.

19. The method of claim 13, further comprising prompting the user to confirm the inclusion of preparation aids, such as bioavailability enhancers, volatility stabilizers, or extraction agents.

20. The method of claim 13, wherein the system adjusts heating, mixing, or potency estimates using stored coefficients for compound interactions.
